(19) United States
(12) Patent Application Publication
(10) Pub. No.: US 2019/0121935 A1
(43) Pub. Date: Apr. 25, 2019

(54) PHENOTYPIC PERSONALIZED MEDICINE: ADAPTIVE OPTIMIZATION OF PATIENT-SPECIFIC COMBINATION THERAPY

(71) Applicant: THE REGENTS OF THE UNIVERSITY OF CALIFORNIA, Oakland, CA (US)

(72) Inventors: Dean HO, Los Angeles, CA (US); Chih-Ming HO, Los Angeles, CA (US); Ali ZARRINPAR, Los Angeles, CA (US); Dong-Keun LEE, Los Angeles, CA (US); Aleidy Marlene Silva VITE, Los Angeles, CA (US); Nakul DATTA, Los Angeles, CA (US)

(73) Assignee: The Regents of the University of California, Oakland, CA (US)

(21) Appl. No.: 15/558,983

(22) PCT Filed: Mar. 17, 2016
(86) PCT No.: PCT/US2016/022932

§ 371 Date: Sep. 15, 2017

Related U.S. Application Data
(60) Provisional application No. 62/135,093, filed on Mar. 18, 2015.

(51) Int. Cl. G16B 20/20; G16H 20/10

(52) U.S. Cl. CPC G06F 19/18; G16H 20/10

(57) ABSTRACT

An initial phenotypic map is derived for a patient subjected to an initial therapeutic regimen including a first drug, based on values of a therapeutic outcome for the patient and corresponding values of a dose of the first drug administered to the patient. Following a change from the initial therapeutic regimen to an updated therapeutic regimen, an updated value of the therapeutic outcome is received for the patient subjected to the updated therapeutic regimen. The initial phenotypic map is re-calibrated according to the updated value of the therapeutic outcome, and, using the re-calibrated phenotypic map, identification is made of a value of the dose of the first drug to be administered to the patient subjected to the updated therapeutic regimen.

### CROSS-REFERENCE TO RELATED APPLICATION

This application claims the benefit of U.S. Provisional Application No. 62/135,093, filed on Mar. 18, 2015, the disclosure of which is incorporated herein by reference in its entirety.

### TECHNICAL FIELD

This disclosure generally relates to optimization of therapy and, more particularly, to optimization of patient-specific combination therapy.

### BACKGROUND

Current challenges with drug delivery include a difficulty in properly identifying an optimized dosing for therapeutic administration for both single drug and combination therapy (e.g., involving two or more drugs) because of a large number of possible drug combinations, dose ratios, drug doses, and so forth, and because of patient heterogeneity, disease heterogeneity, and a host of other factors. Furthermore, conventional approaches relying on genomics or modeling-based approaches for personalized medicine impede the ability to dynamically tune combination or single drug optimization since such conventional approaches are generally not conducive towards dynamic tuning to adjust to varying disease conditions and other changes as a result of resistance and other physiological conditions.

It is against this background that a need arose to develop the embodiments described in this disclosure.

### SUMMARY

In some embodiments, a method includes: (1) deriving an initial phenotypic map for a patient subjected to an initial therapeutic regimen including a first drug, based on values of a therapeutic outcome for the patient and corresponding values of a dose of the first drug administered to the patient; (2) following a change from the initial therapeutic regimen to an updated therapeutic regimen, receiving an updated value of the therapeutic outcome for the patient subjected to the updated therapeutic regimen; (3) re-calibrating the initial phenotypic map according to the updated value of the therapeutic outcome; and (4) using the re-calibrated phenotypic map, identifying a value of the dose of the first drug to be administered to the patient subjected to the updated therapeutic regimen.

In additional embodiments, a method includes: (1) deriving an initial phenotypic map for a patient subjected to an initial therapeutic regimen including a first drug and a second drug, based on values of a phenotypic output for the patient and corresponding values of doses of the first drug and the second drug; (2) following a change from the initial therapeutic regimen to an updated therapeutic regimen, receiving an updated value of the phenotypic output for the patient subjected to the updated therapeutic regimen; (3) re-calibrating the initial phenotypic map according to the updated value of the phenotypic output; and (4) using the re-calibrated phenotypic map, identifying values of the doses of the first drug and the second drug for the patient subjected to the updated therapeutic regimen.

In further embodiments, a method includes: (1) for each patient of a group of patients, deriving parameters of an individual phenotypic map for the patient administered with at least one drug, based on values of a therapeutic outcome for the patient and corresponding values of a dose of the drug administered to the patient; and (2) averaging the parameters across the group of patients to derive averaged parameters of a population level phenotypic map.

Other aspects and embodiments of this disclosure are also contemplated. The foregoing summary and the following detailed description are not meant to restrict this disclosure to any particular embodiment but are merely meant to describe some embodiments of this disclosure.

### BRIEF DESCRIPTION OF THE DRAWINGS

For a better understanding of the nature and objects of some embodiments of this disclosure, reference should be made to the following detailed description taken in conjunction with the accompanying drawings.

FIG. 1: An example of time profiles of drug doses xᵢ(t) (upper panel) and a therapeutic outcome E(t) (lower panel) for the case of a combination of 2 drugs (drug 1 and drug 2) applied to a test subject over the course of multiple treatment cycles, according to an embodiment of this disclosure.

FIG. 2: An example of re-calibration of quadratic phenotypic mapping to account for a regimen change for a patient, according to an embodiment of this disclosure.

FIG. 3: Another example of re-calibration of quadratic phenotypic mapping to account for a regimen change for a patient, according to an embodiment of this disclosure.

FIG. 4: A processing unit implemented in accordance with an embodiment of this disclosure.

FIG. 5: Retrospective clinical analysis using Feedback System Control (FSC). (A) Patient A FSC-optimized and clinically observed tacrolimus serum trough levels are shown (Encircled: FSC, Remainder: Standard). (B) Patient B FSC-optimized and clinically observed tacrolimus serum trough levels are shown (Encircled: FSC, Remainder: Standard). (C) Patient A tacrolimus dosing comparison (Dark dots: FSC, Light dots: Standard). (D) Patient B tacrolimus dosing comparison (Dark dots: FSC, Light dots: Standard). (E) Patient A prednisone dosing comparison (Dark dots: FSC, Light dots: Standard). (F) Patient B prednisone dosing comparison (Dark dots: FSC, Light dots: Standard). (G) Patient A mycophenolate dosing comparison (Dark dots: FSC, Light dots: Standard). (H) Patient B mycophenolate dosing comparison (Dark dots: FSC, Light dots: Standard). (I) Patient A 3-D tacrolimus and mycophenolate drug response map. (J) Patient A 2-D tacrolimus and mycophenolate drug response map. (K) Patient B 3-D tacrolimus and mycophenolate drug response map. (L) Patient B 2-D tacrolimus and mycophenolate drug response map. (M) Patient A 3-D tacrolimus and prednisone drug response map. (N) Patient A 2-D tacrolimus and prednisone drug response map. (0) Patient B 3-D tacrolimus and prednisone drug response map. (P) Patient B 2-D tacrolimus and prednisone drug response map.

FIG. 6: FSC parabolic phenotyping mapping for patient ID5 (target range=6-8 ng/ml). (A) Parabolic mapping for ID5 is shown; R²=0.93 (“−2”, “−1”, “0”, and so forth denote data points obtained on different days). (B) Dual parabolic mapping during recalibration is shown. A deviation from the target range is used to construct a translated parabolic map from which subsequent tacrolimus administration is determined, R²=0.81 (Light open circles), R²=0.28 (Dark dots, due to D18 being within target range). (C) Tacrolimus serum trough levels during the course of FSC-guided treatment is shown. Deviations are attributed to re-calibration following major regimen changes followed by subsequent systematic re-convergence into target ranges. (D) A compensation profile to adjust for hemodialysis by correlating measured changes in trough levels with the length of time between the dialysis procedure and trough level reading. (E) 3-D drug response mapping correlating tacrolimus (mg) and fluconazole dosing (mg) with tacrolimus serum trough levels (ng/ml). (F) 2-D drug response mapping correlating tacrolimus (mg) and fluconazole dosing (mg) with tacrolimus serum trough levels (ng/ml). (G) 3-D drug response mapping correlating tacrolimus (mg) and prednisone dosing (mg) with tacrolimus serum trough levels (ng/ml). (H) 2-D drug response mapping correlating tacrolimus (mg) and prednisone dosing (mg) with tacrolimus serum trough levels (ng/ml).

FIG. 7: FSC parabolic phenotyping mapping for patient ID8 (target range=8-10 ng/ml). (A) Parabolic mapping for ID8 is shown; R²=0.91 (“−2”, “−1”, “0”, and so forth denote data points obtained on different days). (B) Dual parabolic mapping during recalibration is shown. A deviation from the target range is used to construct a translated parabolic map from which subsequent tacrolimus administration is determined, R²=0.96 (Dark dots). (C) Tacrolimus serum trough levels during the course of FSC-guided treatment is shown. Deviations are attributed to re-calibration following major regimen changes followed by subsequent systematic re-convergence into target ranges. (D) 3-D drug response mapping correlating tacrolimus (mg) and mycophenolate dosing (mg) with tacrolimus serum trough levels (ng/ml). (E) 2-D drug response mapping correlating tacrolimus (mg) and mycophenolate dosing (mg) with tacrolimus serum trough levels (ng/ml). (F) 3-D drug response mapping correlating tacrolimus (mg) and prednisone dosing (mg) with tacrolimus serum trough levels (ng/ml). (G) 2-D drug response mapping correlating tacrolimus (mg) and prednisone dosing (mg) with tacrolimus serum trough levels (ng/ml).

FIG. 8: Clinical standard of care profile for control patient ID6 (target range=8-10 ng/ml). (A) Mapping analysis for ID6 is shown (“1”, “2”, “3”, and so forth denote data points obtained on different days). (B) Mapping analysis during recalibration is shown. Incremental dosing increases were plotted, and a linear correlation between tacrolimus dosing and trough level was identified, R²=0.93. (C) Tacrolimus serum trough levels during the course of treatment is shown. (D) 3-D drug response mapping correlating tacrolimus (mg) and mycophenolate dosing (mg) with tacrolimus serum trough levels (ng/ml). (E) 2-D drug response mapping correlating tacrolimus (mg) and mycophenolate dosing (mg) with tacrolimus serum trough levels (ng/ml). (F) 3-D drug response mapping correlating tacrolimus (mg) and prednisone dosing (mg) with tacrolimus serum trough levels (ng/ml). (G) 2-D drug response mapping correlating tacrolimus (mg) and prednisone dosing (mg) with tacrolimus serum trough levels (ng/ml).

FIG. 9: Patient-specific drug effects on tacrolimus serum trough levels. (A) The effects of contrimoxazole administration (left bars) on tacrolimus serum trough levels (right bars) for patients ID1, ID5 and ID7 (Dates of recording provided). (B) The effects of fluconazole administration (left bars) on tacrolimus serum trough levels (right bars) for patients ID1, ID3, and ID7 (Dates of recording provided).

FIG. 10: Systematic FSC-mediated patient mapping and optimization. (A) Patient ID7 drug response map correlating tacrolimus (mg) and cotrimoxazole (mg) with tacrolimus serum trough levels over time representing a synergistic interaction, where highest dosing is not required for target range convergence (5-7 ng/ml). (B) Patient ID7 drug response map correlating tacrolimus (mg) and cotrimoxazole (mg) with tacrolimus serum trough levels over time representing the appearance of an antagonistic interaction. (C) Patient ID7 drug response map correlating tacrolimus (mg) and cotrimoxazole (mg) with tacrolimus serum trough levels over time representing an antagonistic interaction. (D) Patient ID7 drug response map correlating tacrolimus (mg) and cotrimoxazole (mg) with tacrolimus serum trough levels over time representing an antagonistic interaction based on divergent administration conditions that both yield desired endpoints. (E) A comparison between FSC-treated (mean±SD: 1.5±0.58) and control-treated patients (mean±SD: 5.5±4.4) based upon the average number of days that were greater than 2 ng/ml outside of the target trough level (n=4 patients). (F) A comparison between FSC-treated (mean±SD: 0.54±0.08) and control-treated patients (mean±SD: 0.35±0.33) based upon the average area-under-the-curve (AUC) inside of the target range (n=4 patients).

FIG. 11: Time course trough levels for patients ID1-ID8. Serum trough levels for each patient over the course of treatment is shown.

FIG. 12: Clinical summaries for patients ID1-ID8. A summary of anonymous patient demographic information and treatment parameters is provided.

### DETAILED DESCRIPTION

### Feedback System Control (FSC)

Embodiments of this disclosure are directed to identifying optimized inputs for a complex system. The goal of optimization of some embodiments of this disclosure can be any one or any combination of reducing labor, reducing cost, reducing risk, increasing reliability, increasing efficacies, reducing side effects, reducing toxicities, and alleviating drug resistance, among others. In some embodiments, a specific example of administering a biological system with optimized single drug or drug combinations (or combinatorial drugs) is used to illustrate certain embodiments of this disclosure. A biological system can include, for example, an individual cell, a collection of cells such as a cell culture or a cell line, an organ, a tissue, or a multi-cellular organism such as an animal (e.g., a pet or a livestock), an individual human patient, or a group of human patients (e.g., a population or sub-population of human patients). A biological system can also include, for example, a multi-tissue system such as the nervous system, immune system, or cardio-vascular system.

More generally, embodiments of this disclosure can optimize wide varieties of other complex systems by applying pharmaceutical, chemical, nutritional, physical, or other types of stimulations. Applications of embodiments of this disclosure include, for example, optimization of drug combinations, vaccine or vaccine combinations, chemical synthesis, combinatorial chemistry, drug screening, treatment therapy, cosmetics, fragrances, and tissue engineering, as well as other scenarios where a group of optimized system inputs is of interest.

Stimulations (or system inputs) can be therapeutic stimuli to treat diseases, control immunosuppression, or otherwise promote improved health, such as pharmaceutical (e.g., single drug or combinatorial drugs, including existing, generic, and later developed drugs, which are applied towards existing therapeutics, repurposing, and later developed drug optimization), biological (e.g., protein therapeutics, antibody therapeutics, peptide-based therapeutics, hormones, inhibitors, DNA, RNA, or other nucleic acid therapeutics, and immunotherapeutic agents, such as cytokines, chemokines, and immune effector cells such as lymphocytes, macrophages, dendritic cells, natural killer cells, and cytotoxic T lymphocytes), chemical (e.g., chemical compounds, metal-based compounds, ionic agents, and naturally-derived compounds, such as traditional eastern medicine compounds), physical (e.g., light, heat, electrical stimuli, such as electrical current or pulse, and mechanical stimuli, such as pressure, shear force, or thermal energy, such as through use of nanotubes, nanoparticles, or other nanostructures), among others. For example, stimulations (or system inputs) can include air pressure for sleep apnea therapy, where changes in brain response, for example, can serve as system outputs, and system inputs can be modulated air pressure from a continuous positive airway pressure (CPAP) device to dynamically treat apnea during the course of sleep. Imaging agents can be considered as drugs in some embodiments, and these agents can be optimized as well. Examples of imaging agents include magnetic resonance imaging (MM) contrast agents (e.g., gadolinium-based, magnesium sulfate-based, and iron oxide-based, among others), computed tomography (CT) agents, computed axial tomography (CAT) agents, positron emission tomography (PET) agents, near-infrared agents, fluorescent agents, nanotechnology-based agents, glucose, and barium-based agents, among others. Optimization of immunotherapy or chemotherapy regimens are encompassed by this disclosure, such as T-cell immunotherapy (e.g., Chimeric Antigen Receptor (CAR) T-cell therapy, Cytotoxic T Lymphocytes (CTL), anti-programmed death ligand 1 (anti-PD-L1) therapy, anti-programmed death 1 (anti-PD-1) therapy, and associated processes to optimize immunotherapy response such as chemotherapeutic (e.g., combination therapy, monotherapy, or other drug treatment approaches) regimens to modulate lymphocyte levels prior to cell product/therapy administration, among others) and protein and protein fragment-based immunotherapy, among others, with optimized combinations to either promote or sustain T-cell activation against cancer. Other approaches include the development of optimized therapies (combination or monotherapies) to inhibit cytokines or other agents that are produced by a tumor or other mechanisms that may impede immunotherapy efficacy, the production of optimized therapies (combination or monotherapies) to inhibit tumor suppressor cells, and the production of optimized therapies (combination or monotherapies) to optimize the presentation of antigens or other relevant proteins or stimulatory molecules/compounds to enhance the efficacy and safety of immunotherapy. These approaches are applicable towards the optimization of checkpoint inhibition therapy or other relevant cancer vaccine therapies. Furthermore, along with immunotherapy or chemotherapy regimens, rapid optimization of drug therapy in concert with such regimens can be attained as well. For example, T-cell immunotherapy with optimized drug combinations can be applied to optimize therapeutic efficacy and safety. In addition, T-cell immunotherapy with optimized combinations of various compounds can be used to optimize T-cell activation to improve treatment efficacy and safety. Moreover, veterinary therapeutic agents can be optimized in some embodiments.

In the case of drugs, for example, drug release can be administered systemically via any one or any combination of intravenous, oral, intramuscular, intraperitoneal, via eye drops, transdermal, via ointments/creams, and via a medical device (e.g., pump infusion, implantable, transdermal, ocular, nasal, otological, oral cavity, and so forth).

Diseases can include, for example, cancer, cardiovascular diseases, pulmonary diseases, atherosclerosis, diabetes, metabolic disorders, sleep disorders (e.g., apnea), genetic diseases, viral diseases (e.g., human immunodeficiency virus, hepatitis B virus, hepatitis C virus, and herpes simplex virus-1 infections), bacterial diseases, and fungal diseases, among others. Some embodiments of this disclosure are implemented and validated in a clinical setting to optimize immunosuppression, but the optimization technique can be extended towards other disorders and health related applications, such as cancers, infectious diseases, nutraceuticals, herbal, or eastern medication, homeopathic treatment, cosmetics, immunotherapy and immunomodulation, and probiotic optimization, among others. More generally, the optimization technique of embodiments of this disclosure is applicable towards virtually all classes of diseases, since the diseases mediate phenotypic change which is an output that the optimization technique uses to realize optimal therapeutic outcomes. Optimization can include complete optimization in some embodiments, but also can include substantially complete or partial optimization in other embodiments.

Stimulations can be applied to direct a complex system towards a desired state, such as applying drugs to treat a human patient having a disease, or to control immunosuppression of a human patient following organ transplantation. The types and characteristics of the stimulations are part of system inputs that can affect the efficiency in bringing the system towards the desired state, where the characteristics of the stimulations can include their amplitudes (e.g., drug doses or dose ratios). However, m types of different drugs with n possible doses for each drug will result in n^{m} possible drug-dose combinations. To identify an optimized or even near optimized combination by multiple tests on all possible combinations is prohibitive in practice. For example, it is not practical to perform all possible drug-dose combinations in animal and clinical tests for finding an effective drug combination as the number of drugs and doses increase.

In some embodiments, a FSC optimization technique allows a rapid search for optimized combinations of system inputs to guide multi-dimensional (or multi-variate) engineering, medicine, financial, and industrial problems, as well as controlling other complex systems with multiple inputs toward their desired states. An optimization technique can be used to identify at least a subset, or all, optimized combinations or sub-combinations of inputs that produce desired states of a complex system. Taking the case of combinational drugs, for example, a combination of m drugs can be evaluated to rapidly identify optimized doses of the m drugs, where m is greater than 1, such as 2 or more, 3 or more, 4 or more, 5 or more, 6 or more, 7 or more, 8 or more, 9 or more, or 10 or more. The optimization technique also can be used to optimize a single drug administration, such that m, more generally, can be 1 or greater than 1.

In some embodiments, an outcome of a complex system in response to multiple inputs can be represented by a low order equation, such as a second order (or quadratic) equation, although a first order (or linear) equation as well as a third order (or cubic) equation are also contemplated as possible low order equations. Also, higher order equations are contemplated for other embodiments. Taking the case of combinational drugs, for example, a therapeutic outcome E can be represented as a function of drug doses as follows:

$$E ( t ) = {E}_{0} + {\sum }_{i} {a}_{i} {x}_{i} ( t ) + {\sum }_{{ii}^{′}} {b}_{{ii}^{′}} {x}_{i} ( t ) {x}_{{i}^{′}} ( t ) + higher order terms \qquad ( 1 )$$

where E(t) is the time-varying therapeutic outcome (e.g., drug efficacy and optionally one or more additional optimization criteria) for a test subject (e.g., a human patient) at time t, E₀ is a parameter (e.g., a constant) corresponding to a baseline therapeutic outcome (e.g., without application of drugs) for the test subject, xᵢ(t) is a time-varying concentration or dose for the test subject (e.g., an external dose as administered to the test subject or an internal dose within the test subject, such as a drug blood, saliva, or serum level) of an i^{th} drug at time t, aᵢ is a parameter (e.g., a constant) corresponding to a first order transfer function between the therapeutic outcome and the i^{th} drug, bᵢᵢ, is a parameter (e.g., a constant) corresponding to a second order transfer function between the therapeutic outcome and the i^{th} and i′^{th} drugs representing drug-drug interaction, and the summations run through m corresponding to the total number of drugs in a drug combination being evaluated. It is also contemplated that a similar equation as equation (1) can be used to represent the therapeutic outcome E(t) as a function of cumulative concentrations or doses (e.g., an integration of the drug dose xᵢ(t) over time, such as an integration of a drug blood or serum level over time using any appropriate method which may include determining an area under a curve to a specific point in time t, as well as other relevant approaches), and an optimization technique can be similarly applied as explained below.

If cubic and other higher order terms are omitted, then the therapeutic outcome E(t) can be represented by a quadratic function of the drug doses xᵢ(t). As noted above, other representations, including ternary and higher order equations or the use of a linear regression representation, are also contemplated. Also, although a specific example of combinational drugs is used, it should be noted that the above equation (1) more generally can be used to represent a wide variety of other complex systems as a function of multiple system inputs.

In some embodiments, the therapeutic outcome E(t) can be measured or derived as a weighted combination or a weighted sum of optimization criteria as follows:

$$E ( t ) = {\sum }_{k = 1}^{o} [ {w}_{k} \times {OC}_{k} ( t ) ] \qquad ( 2 )$$

where OCₖ(t) is a k^{th} optimization criterion for the test subject at time t, wₖ is a weighting factor that can be adjusted or tuned to determine a relative weight of OCₖ(t) in optimizing the therapeutic outcome E(t), o is a total number of different optimization criteria being evaluated, and o is 1 or greater than 1, such as 2 or more, 3 or more, 4 or more, 5 or more, 6 or more, 7 or more, 8 or more, 9 or more, or 10 or more. In some embodiments, a sum of all weighting factors is 1 (e.g., w₁+w₂ . . . +w₀=1), although a value of this sum can be varied for other embodiments. In addition to the above equation (2), other representations of the therapeutic outcome E(t) are contemplated and encompassed by this disclosure.

Taking the case of combinatorial drugs, for example, OCₖ(t) is the k^{th} optimization criterion in the design of the combination of m drugs. Examples of optimization criteria include drug efficacy, drug toxicity, drug safety, drug side effects, drug tolerance, therapeutic window, and drug cost, among others. In the above equation (2), the therapeutic outcome E(t) represents an overall outcome or response to be optimized (e.g., reduced or minimized, or enhanced or maximized), and is a weighted sum of the o different optimization criteria. In some embodiments, at least one of the o different optimization criteria can correspond to a phenotypic response of the test subject that is subjected to the combination of m drugs. For example, at least one optimization criterion can correspond to drug efficacy, and at least another optimization criterion can correspond to drug safety or toxicity. An optimization criterion can directly correspond a phenotypic response of the test subject, or can be calculated or otherwise derived from one or more phenotypic responses, such as by applying proper transformations to adjust a range and scale of the phenotypic responses.

Certain phenotypic responses are desirable, such as drug efficacy or drug safety, while other phenotypic responses are undesirable, such as drug toxicity or drug side effects. In the case of the latter phenotypic responses, their weighting factors serve as penalty factors in the optimization of the combination of m drugs. Various weighting factors in the above equation (2) can be adjusted or tuned to reflect the relative importance of desirable optimization criteria and undesirable optimization criteria, and the adjustment or tuning can be performed on a case-by-case basis to yield different optimized doses of the m drugs depending on the particular test subject. Also, the adjustment or tuning of the weighting factors can be performed over time so as to incorporate feedback over the course of a treatment.

A phenotypic response of a test subject can include, or can be calculated or otherwise derived from, pharmacodynamics data, such as related to quantitative measurements or readouts of markers of treatment response. Alternatively, or in combination, a phenotypic response of a test subject can include qualitative measurements or readouts of treatment response, which can be graded or evaluated on a scale. Examples of measurements or readouts of phenotypic responses include:

(1) Use of hair, fecal matter, sweat, mucus, cheek swabs, earwax, tears, sperm, skin cells or scrapes, and other excretions or biological materials to screen for markers for tumor treatment response, including proteins and protein fragments, cell, blood, and nucleic acids (e.g., small interfering RNA (siRNA), microRNA (miRNA), long noncoding RNA, DNA, exosomes, and other classes of ribosomal and deoxyribosomal nucleic acids);

(2) Patient body temperature, blood pressure, pupil dilation, body weight, fluid intake or brain waves, electrochemical readings of the brain, cardiac signals, excretion, and palpation;

(3) Blood draws to monitor levels of circulating tumor markers (e.g., cytokines, antibodies, serum proteins, electrolytes, hematocrit levels, and general protein and biological markers) that serve as indicators for tumor treatment response;

(4) Urine analysis to monitor levels of electrolyte, protein, possible presence of blood, or other markers that serve as indicators for tumor treatment response—additional markers include proteins and protein fragments, cell, and nucleic acids (e.g., siRNA, miRNA, long noncoding RNA, DNA, exosomes, and other relevant nucleic acids);

(5) Sputum analysis to assess number of sperms for infertility treatment and for relevant markers associated with tumor treatment response (e.g., proteins and protein fragments, cell, blood, and nucleic acids, such as siRNA, miRNA, long noncoding RNA, DNA, exosomes, and other classes of ribosomal and deoxyribosomal nucleic acids);

(6) Saliva analysis to assess for relevant markers associated with tumor treatment response (e.g., proteins and protein fragments, cell, blood, and nucleic acids, such as siRNA, miRNA, long noncoding RNA, DNA, exosomes, and other classes of ribosomal and deoxyribosomal nucleic acids);

(7) Use of imaging techniques, such as X-ray, PET, CT, CAT, MRI (e.g., conventional MM, functional MRI, or other types of MRI), fluorescence spectroscopy, near-infrared spectroscopy, Raman spectroscopy, fluorescence correlation spectroscopy, acoustic imaging techniques, microscopy of tissue, biopsy, and other imaging techniques to monitor tumor size or to monitor fluid and blood flow to and from a tumor as an indicator for tumor treatment response, or blood flow to and from an area of the body (e.g., brain, heart, and so forth) as an indicator of general treatment response;

(8) Image processing techniques to quantify tumor treatment response from imaging techniques (e.g., pixel counting, heat maps, or other techniques)—image processing techniques also can include image analysis for hematoxylin and eosin staining or other cell or tissue stains to quantify tumor response, fluorescent marker quantification to assess tumor response, and quantification of biopsy (e.g., fine needle aspiration) samples and other relevant biological materials to quantify tumor treatment response; and

(9) Skin analysis for accessing color, lipid, and blood circulation for cosmetic treatments.

Referring back to equation (1), for the case of m=1 (a total of 1 drug), then:

E(t)=E₀+a₁x₁(t)+b₁₁x₁(t)x₁(t) (3)

with a total of three parameters, E₀, a₁, and b₁₁.

For the case of m=2 (a total of 2 drugs), then:

E(t)=E₀+a₁x₁(t)+a₂x₂(t)+b₁₂x₁(t)x₂(t)+b₁₁x₁(t)x₂(t)+b₂₂x₂(t)x₂(t) (4)

with a total of six parameters, E₀, a₁, a₂, b₁₂, b₁₁, and b₂₂.

More generally form total drugs, a total number of parameters p is 1+2m+(m(m−1))/2. If one drug dose is kept invariant in the study, the number of parameters p can be further reduced to 1+2(m−1)+((m−1)(m−2))/2, for m>1. Table 1 below sets forth a total number of parameters in a quadratic function of the therapeutic outcome with respect to a total number drugs being evaluated.

TABLE 1

| | | Parameters (p) |
|---|---|---|
| | | (if one drug dose |
| Drugs (m) | Parameters (p) | is kept invariant) |
| 1 | 3 | — |
| 2 | 6 | 3 |
| 3 | 10 | 6 |
| 4 | 15 | 10 |
| 5 | 21 | 15 |
| 6 | 28 | 21 |

Advantageously, a small number of measurements or readouts of drug doses and phenotypic responses can be performed over time, and results of the measurements or readouts can be received and used to represent a therapeutic outcome-dose response surface, such as a quadratic phenotypic map, and this input/output response surface can be used to identify optimized drug-dose combinations. Also, by measuring or deriving the time course variations of the drug doses and the phenotypic responses, the number of test subjects can be minimized or reduced, even down to one, thereby realizing personalized medicine or phenotypic personalized medicine in a clinical setting.

Taking the case of the quadratic function of the therapeutic outcome E(t), for example, multiple measurements or readouts of the drug doses and the therapeutic outcome can be performed over time for the test subject as follows:

$$\begin{matrix}E ( {t}_{1} ) = {E}_{0} + {\sum }_{i} {a}_{i} {x}_{i} ( {t}_{1} ) + {\sum }_{{ii}^{′}} {b}_{{ii}^{′}} {x}_{i} ( {t}_{1} ) {x}_{{i}^{′}} ( {t}_{1} ) & ( 5 ) \\ E ( {t}_{2} ) = {E}_{0} + {\sum }_{i} {a}_{i} {x}_{i} ( {t}_{2} ) + {\sum }_{{ii}^{′}} {b}_{{ii}^{′}} {x}_{i} ( {t}_{2} ) {x}_{{i}^{′}} ( {t}_{2} ) & \\ … & \\ E ( {t}_{j} ) = {E}_{0} + {\sum }_{i} {a}_{i} {x}_{i} ( {t}_{j} ) + {\sum }_{{ii}^{′}} {b}_{{ii}^{′}} {x}_{i} ( {t}_{j} ) {x}_{{i}^{′}} ( {t}_{j} ) & \end{matrix}$$

where E(tⱼ) is the therapeutic outcome measured or derived at time tⱼ from a total of q measurement instances, and xᵢ(tⱼ) is the dose of the i^{th} drug measured or derived at time tⱼ from the total of q measurement instances. From the q measurement instances, the p parameters E₀, aᵢ, and aᵢⱼ can be derived, with q≥p, namely with the number of measurement instances being the same as, or greater than, the number of parameters in the quadratic function of some embodiments. In some embodiments, a reduced number of measurement instances can be conducted, such as with q=p. If one drug dose is kept invariant in the study, the number of measurement instances q can be further reduced to 1+2(m−1)+((m−1)(m−2))/2, for m>1. Also, in some embodiments, the number of measurement instances q can be even further reduced, by using interpolation to derive one or more therapeutic outcome values from measured therapeutic outcome values, by using interpolation to derive one or more dose values from measured dose values, or both.

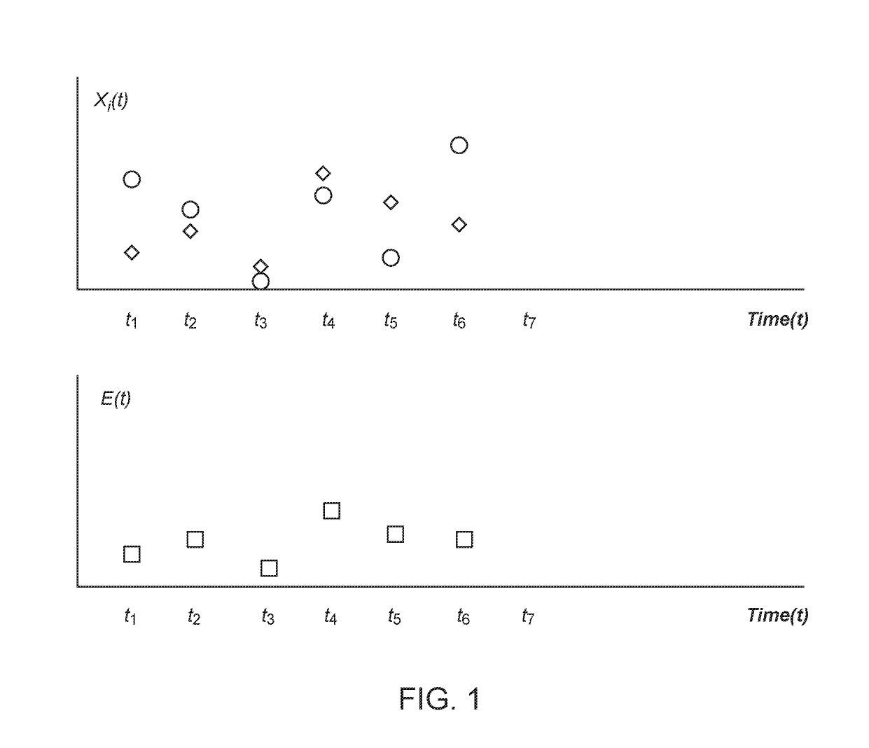

FIG. 1 shows an example of time profiles of drug doses xᵢ(t) (upper panel) and a therapeutic outcome E(t) (lower panel) for the case of a combination of 2 drugs (drug 1 and drug 2) applied to a test subject over the course of multiple treatment cycles, according to an embodiment of this disclosure. Doses for drug 1 are represented by circles, while doses for drug 2 are represented by diamonds. In this example, the optimization technique is applied to identify optimized doses of the 2 drugs that are individually tailored for the test subject and are applied to the test subject in subsequent treatment cycles, based on results of measurements performed on the test subject during a calibration period including one or more initial treatment cycles. Although the example of 2 drugs is explained with reference to FIG. 1, it will be understood that the optimization technique can be applied to a number of drugs more or less than 2.

Referring to FIG. 1, during the calibration period, values of the doses xᵢ(t) for drug 1 and drug 2 are measured or obtained at multiple measurement instances, here 6 values each for drug 1 and drug 2 at t₁ through t₆. Also during the calibration period, values of the therapeutic outcome E(t) are measured or obtained at multiple measurement instances, here 6 values at t₁ through t₆. Although this example sets forth 6 measurement instances of the drug doses and 6 measurement instances of the therapeutic outcome, less than 6 measurement instances can be performed for either, or both, the drug doses and the therapeutic outcome, with remaining values derived from a reduced set of measured values through interpolation. It is also contemplated that the drug doses xᵢ(t) can be external doses as administered to the test subject at multiple administration instances, and measurements of the therapeutic outcome E(t) can be performed at multiple measurement instances having a time lag or delay relative to the administration instances, such that, for example, the time axis in the lower panel is shifted relative to the time axis in the upper panel.

Once measurements are performed on the time course variations of stimulations and an outcome of a complex system in response to the time-varying stimulations, experimental results of the measurements are then fitted into a response surface or map of the system by using multi-dimensional fitting, such as regression analysis. Based on the fitting performance between the experimental results and the map, additional measurements can be conducted to improve the accuracy of the map. Once the map with a desired accuracy is achieved, optimized combinations of the stimulations and their optimized characteristics can be identified by using a suitable extrema locating technique, such as by locating global or local maxima in a response surface. Taking the case of the quadratic phenotypic map of the therapeutic outcome E(t), for example, optimized doses can be identified once the parameters E₀, aᵢ, and bᵢⱼ are derived through multi-dimensional fitting:

$${E}_{opt} ( t ) = {E}_{0} + {\sum }_{i} {a}_{i} {x}_{i , opt} ( t ) + {\sum }_{{ii}^{′}} {b}_{{ii}^{′}} {x}_{i , opt} ( t ) {x}_{{i}^{′} , opt} ( t ) \qquad ( 6 )$$

where x_{i,opt}(t) is an optimized dose of the i^{th} drug applied to the test subject at time t.

Referring back to the example of FIG. 1, the 6 parameters E₀, a₁, a₂, b₁₂, b₁₁, and b₂₂ of the quadratic map of the therapeutic outcome E(t) can be derived from the 6 measured or obtained values of the drug doses x₁(t) at t₁ through t₆ and the 6 measured or obtained values of the therapeutic outcome E(t) at t₁ through t₆. Using the quadratic map of the therapeutic outcome E(t), optimized doses of drug 1 and drug 2 can be identified, and the optimized doses can be applied to the test subject at a next treatment cycle, here at time t₇. The quadratic map of the therapeutic outcome and the optimized doses of drug 1 and drug 2 can be continually updated over the course of treatment using a moving time window approach, such that time-varying phenotypic responses of the test subject can be accommodated, and the drug doses can be optimized according to the latest or current phenotype of the test subject.

### Retrospective Optimization to Derive Patient-Specific Sensitivity Data to Compensate for Therapeutic Regimen Changes and Further Optimize Therapy

In some embodiments, the FSC optimization technique explained above can be implemented on prior patient-specific data in a retrospective fashion, where the prior data can be used to retrospectively optimize treatment to obtain drug-dose combination information for both personalized medicine and optimal drug design. In addition, this approach can be used to obtain information pertaining to patient sub-population-specific characteristics that can provide dosing compensation strategies, such as understanding patient sub-population trends following regimen changes or new drugs being introduced into treatment, understanding antagonistic, synergistic, or additive interactions among drugs to compensate for regimen changes, and so forth. For example, certain patients may have drug efficacy or serum level increases or decreases depending on an increase or a decrease in dose of another drug that is part of a multi-therapeutic regimen. These patient or population-specific drug sensitivity levels can be retrospectively (or prospectively) determined to assess whether such levels are inversely or directly related (e.g., antagonistic or synergistic) on a patient-specific, population-specific, or sub-population-specific level. This sensitivity information can aid with prospectively compensating for anticipated changes to therapeutic efficacy and safety to even further optimize therapeutic outcomes. A combination of personalized data and population or sub-population level data can improve the optimization while reducing effort and time in attaining enhanced efficacy and safety of treatment.

### Prospective Optimization to Compensate for Therapeutic Regimen Changes and Simultaneously Manage Co-Infections, Parallel Procedures, and Conditions Other than a Primary Condition

In some embodiments, the FSC optimization technique can be implemented to allow modulation/rational management of a subset of drugs (e.g., modulation in administration of a single drug) within a multi-therapeutic regimen to optimize treatment via shifting and re-calibration of quadratic phenotypic mapping in response to changes in regimen. Using this approach, when a change in regimen is made to another drug or procedure (other than a particular drug or a particular subset of drugs being modulated), a translation process or a sensitivity adjustment can be employed where a quadratic phenotypic map can be shifted according to a deviation from a target therapeutic outcome. In some embodiments, one data point may be adequate to initially re-calibrate a patient's response, thereby allowing accurate and rapid re-converging into a target therapeutic outcome by re-constructing an updated quadratic phenotypic map for continued optimization. Additional data points can be used to re-converge into the target therapeutic outcome as desired. Using this approach, one drug among a multi-therapeutic regimen can be modulated to effectively optimize patient-specific therapy, even with changes in administration of other drugs or procedures to treat co-existing conditions such as infections and other disorders. In such manner, the FSC optimization technique allows for adaptable personalization of treatment as it is a highly actionable platform. It is capable of adjusting to changes in regimens while accounting for multiple simultaneous conditions that may accompany a primary condition.

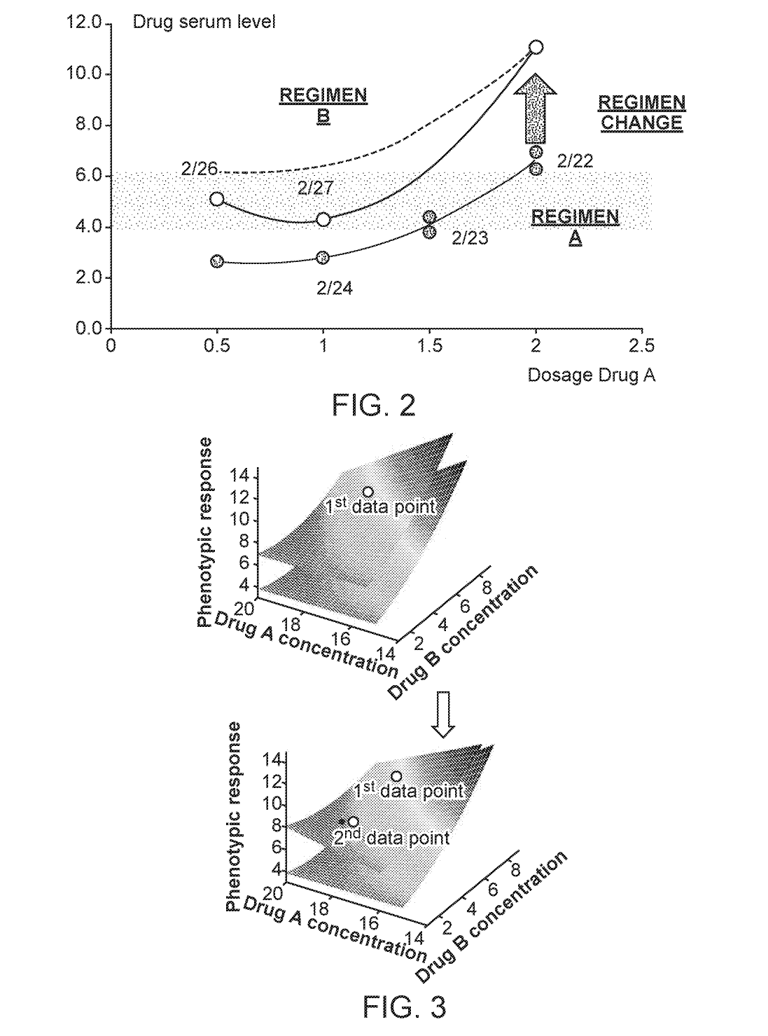

FIG. 2 shows an example of re-calibration of quadratic phenotypic mapping to account for a regimen change for a patient. The patient is administered with a multi-drug combination of Drug A to treat a primary condition as well as Drugs B and C to treat secondary conditions such as infection, inflammation, or other disorders. One or more procedures (e.g., hemodialysis and various drug formulations, such as suspension and tablet) also can be included as part of the patient's regimen, and changes to such procedures also can be accounted as part of the re-calibration. Without modulating or directing the dose of every drug within the combination, a therapeutic goal in this example is to converge a drug serum level within a certain range, as illustrated in FIG. 2 within a target range of 4-6 ng/ml, by modulating the dose of Drug A alone.

Referring to FIG. 2, the patient is initially subjected to regimen A during a time period encompassing Days 2/22, 2/23, and 2/24, and, during a calibration period while under regimen A, an initial quadratic phenotypic map is derived for the patient (shown as a lower solid curve) based on at least three data points corresponding to values of the drug serum level for at least three measurement instances. Using the initial quadratic phenotypic map, optimized doses of Drug A can be identified such that the drug serum level can be maintained within the target range of 4-6 ng/ml.

When a regimen change occurs on Day 2/25 from regimen A to regimen B (e.g., reducing or increasing the dose of Drug B or C, adding a new Drug D to the combination, starting a parallel procedure, and so forth), re-calibration is conducted to compensate for changes to the drug serum level, such as arising from drug-drug interactions that can cause a shift or deviation in the drug serum levels away from the target range. On the day of the regimen change, namely on Day 2/25, sensitivity information from retrospective analysis using FSC can aid with compensating for anticipated changes to the drug serum level, such as by guiding the dose of Drug A that is administered to the patient on Day 2/25 while accounting for antagonistic or synergistic interactions among drugs from the retrospective analysis. Once a data point is obtained for the drug serum level following the regimen change on Day 2/25, re-calibration can be performed by shifting the initial quadratic phenotypic map according to that data point, as shown in FIG. 2 by the dashed curve. Such shifting can be performed so that the shifted quadratic phenotypic map intersects the initial data point following the regimen change, while maintaining a shape or curvature of the initial quadratic phenotypic map. While full re-construction of an updated quadratic phenotypic map would involve at least three data points under regimen B, the shifted quadratic phenotypic map allows identification of an approximation of where a subsequent dose of Drug A should reside on Day 2/26, prior to full re-construction of the updated quadratic phenotypic map while re-converging the drug serum level towards the target range. Here in this example, a dose of “0.5” of Drug A is identified according to the shifted quadratic phenotypic map, and the identified dose of Drug A can be administered to the patient on Day 2/26, prior to full re-construction of the updated quadratic phenotypic map. Thus, rapid re-calibration under the regimen change can be performed on the basis of a total of just four data points from just four measurement instances (three data points to derive the initial quadratic phenotypic map, and the initial data point obtained following the regimen change), as compared to a total of six data points from six measurement instances (three data points to derive the initial quadratic phenotypic map, and three data points to derive the updated quadratic phenotypic map following the regimen change). Once at least three data points are obtained for the patient while under regimen B, the updated quadratic phenotypic map is derived for the patient (shown as an upper solid curve), and optimized doses of Drug A can be identified using the updated quadratic phenotypic map. Re-calibration under further regimen changes can be performed in a similar manner.

FIG. 3 shows another example of re-calibration of quadratic phenotypic mapping to account for a regimen change for a patient. The patient is administered with a multi-drug combination of Drugs A and B to treat a primary condition as well as Drugs C and D to treat secondary conditions such as infection, inflammation, or other disorders. One or more procedures (e.g., hemodialysis and various drug formulations, such as suspension and tablet) also can be included as part of the patient's regimen, and changes to such procedures also can be accounted as part of the re-calibration. Without modulating or directing the dose of every drug within the combination, a therapeutic goal in this example is to converge a phenotypic response or output within a certain target range, by modulating the doses of Drugs A and B alone.

Referring to FIG. 3, the patient is initially subjected to regimen A, and, during a calibration period while under regimen A, an initial quadratic phenotypic map is derived for the patient (shown as a lower quadratic surface (e.g., a lower paraboloid surface)) based on at least six data points corresponding to values of the phenotypic response for at least six measurement instances. Using the initial quadratic phenotypic map, optimized doses of Drugs A and B can be identified such that the phenotypic response can be maintained within the target range.

When a regimen change occurs from regimen A to regimen B (e.g., reducing or increasing the dose of Drug C or D, adding a new Drug E to the combination, starting a parallel procedure, and so forth), re-calibration is conducted to compensate for changes to the phenotypic response, such as arising from drug-drug interactions that can cause a shift or deviation in the phenotypic response away from the target range. On the day of the regimen change, sensitivity information from retrospective analysis using FSC can aid with compensating for anticipated changes to the phenotypic response, such as by guiding the dose of Drug A or Drug B that is administered to the patient on that day while accounting for antagonistic or synergistic interactions among drugs from the retrospective analysis. Once a data point is obtained for the phenotypic response following the regimen change (shown in FIG. 3 as “1^{st} data point”), re-calibration can be performed by shifting the initial quadratic phenotypic map according to that data point, as shown in the left panel of FIG. 3 by an upper quadratic surface (e.g., an upper paraboloid surface). Such shifting can be performed so that the shifted quadratic phenotypic map intersects the initial data point following the regimen change, while maintaining a shape or curvature of the initial quadratic phenotypic map. While full re-construction of an updated quadratic phenotypic map would involve at least six data points under regimen B, the shifted quadratic phenotypic map allows identification of an approximation of where subsequent doses of Drugs A and B should reside on the next treatment instance following the regimen change, prior to full re-construction of the updated quadratic phenotypic map while re-converging the phenotypic response towards the target range. Thus, rapid re-calibration under the regimen change can be performed on the basis of a total of just seven data points from just seven measurement instances (six data points to derive the initial quadratic phenotypic map, and the initial data point obtained following the regimen change), as compared to a total of twelve data points from twelve measurement instances (six data points to derive the initial quadratic phenotypic map, and six data points to derive the updated quadratic phenotypic map following the regimen change). Once at least six data points are obtained for the patient while under regimen B, the updated quadratic phenotypic map is derived for the patient (shown as an upper quadratic surface (e.g., an upper paraboloid surface) in the right panel of FIG. 3), and optimized doses of Drugs A and B can be identified using the updated quadratic phenotypic map. Re-calibration under further regimen changes can be performed in a similar manner.

Thus, where a therapeutic goal is to converge a phenotypic response or output by modulating a dose of a single drug within a multi-therapeutic regimen, rapid re-calibration under a regimen change can be performed on the basis of a total of just four data points from just four measurement instances, as compared to a total of six data points from six measurement instances otherwise involved for a full re-construction of an updated quadratic phenotypic map. And, where a therapeutic goal is to converge a phenotypic response or output by modulating doses of two drugs within a multi-therapeutic regimen, rapid re-calibration under a regimen change can be performed on the basis of a total of just seven data points from just seven measurement instances, as compared to a total of twelve data points from twelve measurement instances otherwise involved for a full re-construction of an updated quadratic phenotypic map. And, where a therapeutic goal is to converge a phenotypic response or output by modulating doses of three drugs within a multi-therapeutic regimen, rapid re-calibration under a regimen change can be performed on the basis of a total of just eleven data points from just eleven measurement instances, as compared to a total of twenty data points from twenty measurement instances otherwise involved for a full re-construction of an updated quadratic phenotypic map.

### Quadratic Phenotypic Mapping to Optimize Therapy, Such as Treatment of Simultaneous Disorders

In some embodiments, another capability of the FSC optimization technique involves the implementation on regimens that are anticipated to be administered for a patient. Specifically, a projected quadratic phenotypic map can be derived such that drug administration thresholds or limits can be determined in advance of treatment, and dosing compensation can be determined in advance to prevent the spiking or sudden drops in therapeutic endpoints or drug levels. This approach effectively allows for barriers or upper and lower limit criteria to be implemented in advance to further improve treatment.

### Population-Personalized Medicine

By harnessing the FSC technique for phenotypic personalized medicine, an approach can be developed to mediate population-personalized medicine. In some embodiments, individually derived parameters (e.g., constants) of a quadratic phenotypic map can be identified across a group of patients using FSC, and the individually derived parameters can be averaged or otherwise combined to arrive at averaged parameters of a quadratic phenotypic map, which, in turn, can be used to derive population-optimized doses. The population-optimized doses can be used for population-based administration of a single drug or drug combinations to either complement personalized or individualized regimens or serve as standalone optimized administration that is tailored for populations or sub-populations of patients.

Using modulation of a single drug as an example, if patient A's individually derived quadratic phenotypic map is y=32.95−11.69x+1.22x², and patient B's individually derived quadratic phenotypic map is y=20.3−6.4x+0.73x², then a population-level quadratic phenotypic map using these two patients as a representative population would be a parabolic function with constants derived by averaging the individually derived constants, namely y=26.625−9.045x+0.975x².

In some embodiments, averaging of individually derived parameters across a group of patients can be a straight or un-weighted average across the group of patients, namely where values of individually derived parameters carry the same weight when averaging. In other embodiments, averaging of individually derived parameters across a group of patients can be a weighted average, where values of individually derived parameters across different sub-groups of patients can carry respective and potentially different weights when averaging. In such embodiments, the different sub-groups of patients can represent or correspond to different sub-populations of patients having characteristics that can affect, influence, or correlate to varying responses to a disease or a treatment of a disease or other condition. Examples of sub-groups of patients include those categorized according to weight, gender, race, and age, among other categories, as well as combinations and sub-combinations of the foregoing categories. Thus, for example, when deriving a population-averaged parameters of a quadratic phenotypic map, values of individually derived parameters for one sub-group of patients can be assigned a higher weight than values of individually derived parameters for another sub-group of patients. In other embodiments, averaging of individually derived parameters can be separately performed for each sub-group of patients to arrive at respective sub-population-averaged parameters for different sub-groups of patients. Thus, for example, values of individually derived parameters for one sub-group of patients can be averaged to arrive at averaged parameters specific for that sub-group of patients as one representative sub-population, values of individually derived parameters for another sub-group of patients can be averaged to arrive at averaged parameters specific for that sub-group of patients as another representative sub-population, and so forth.

### Processing Unit

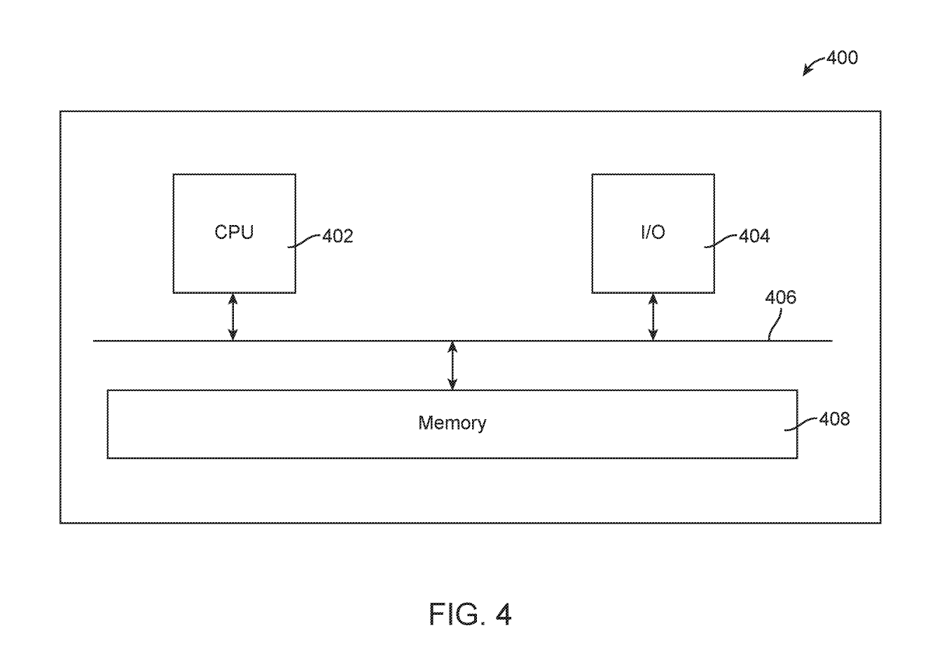

FIG. 4 shows a processing unit 400 implemented in accordance with an embodiment of this disclosure. Depending on the specific application, the processing unit 400 can be implemented as, for example, a portable electronic device, a client computer, or a server computer. Referring to FIG. 4, the processing unit 400 includes a central processing unit (CPU) 402 that is connected to a bus 406. Input/output (I/O) devices 404 are also connected to the bus 406, and can include a keyboard, mouse, display, and the like. An executable program, which includes a set of software modules for certain operations described in this disclosure, is stored in a memory 408, which is also connected to the bus 406. The memory 408 can also store a user interface module to generate visual presentations.

An embodiment of this disclosure relates to a non-transitory computer-readable storage medium having computer code thereon for performing various computer-implemented operations. The term “computer-readable storage medium” is used herein to include any medium that is capable of storing or encoding a sequence of instructions or computer codes for performing the operations described herein. The media and computer code may be those specially designed and constructed for the purposes of this disclosure, or they may be of the kind well known and available to those having skill in the computer software arts. Examples of computer-readable storage media include, but are not limited to: magnetic media such as hard disks, floppy disks, and magnetic tape; optical media such as CD-ROMs and holographic devices; magneto-optical media such as floptical disks; and hardware devices that are specially configured to store and execute program code, such as application-specific integrated circuits (ASICs), programmable logic devices (PLD s), and ROM and RAM devices. Examples of computer code include machine code, such as produced by a compiler, and files containing higher-level code that are executed by a computer using an interpreter or a compiler. For example, an embodiment of this disclosure may be implemented using Java, C++, or other object-oriented programming language and development tools. Additional examples of computer code include encrypted code and compressed code. Moreover, an embodiment of this disclosure may be downloaded as a computer program product, which may be transferred from a remote computer (e.g., a server computer) to a requesting computer (e.g., a client computer or a different server computer) via a transmission channel. Another embodiment of this disclosure may be implemented in hardwired circuitry in place of, or in combination with, machine-executable software instructions.

### Example

The following example describes specific aspects of some embodiments of this disclosure to illustrate and provide a description for those of ordinary skill in the art. The example should not be construed as limiting this disclosure, as the example merely provides specific methodology useful in understanding and practicing some embodiments of this disclosure.

### Optimizing Liver Transplant Immunosuppression Using a Phenotypic Personalized Medicine Platform

Overview

Immunosuppressive drugs such as tacrolimus have narrow therapeutic target ranges following liver transplantation. Therefore, inter-individual and intra-individual variability in dosing requirements conventionally result in titrated drug administration that results in frequent deviation from target ranges, particularly during the critical post-operative phase. Previous studies have sought to identify genetic and medical factors that impact tacrolimus levels. However, rapid calibration to fully assess and effectively respond to individualized responses to tacrolimus administration as well as other post-operative drugs such as mycophenolate (immunosuppressant), prednisone (steroid), and a host of prophylactic antibiotics has thus far not been achieved. To address this challenge, this example sets forth the development of a mechanism-independent and model-less phenotypic personalized medicine platform (PPM3′) that can calibrate patients on an individualized level, and tailor therapy towards a broad spectrum of diseases. PPM3′ was developed to rationally reconcile phenotypic responses to therapeutic intervention to prescribe optimized and patient-specific therapeutic regimens. In this example, PPM3′ successfully identified patient-specific therapeutic response constants, or indicators of how that specific patient's serum drug levels would respond to multi-drug therapy for immunosuppression and co-infection. These constants were then utilized to mediate parabolic phenotypic mapping to rationally guide clinical tacrolimus administration. Importantly, PPM3′ showed that drug antagonism or synergism were patient-specific, and directed formulation of adaptive dosing compensation strategies by taking anti-inflammation/antifungal therapy and procedures such as hemodialysis into account, and subsequently prescribed systematically determined immunosuppressive therapy. This resulted in a personalized approach to avoid or minimize target range deviation, clinically validating the approach as a powerful phenotypic personalized medicine platform.

Introduction

Improvements in post-transplant survival have been made largely due to more selective and less toxic immunosuppression regimens and advances in operative and perioperative care. An increase in the number of available immunosuppressive agents and formulations, a more complete understanding of their molecular mechanisms, improved protocols in therapeutic drug monitoring, and refinement of targeted therapeutic ranges have all played major roles in this advance. What remains is the promise of individualized immunosuppressive therapy with the goal of further increasing patient and graft function and survival, while reducing rejection and toxicity. It appears, however, patients will still be administered with a large number of therapeutic and prophylactic medications, each with its own distinct pharmacologic and metabolic profile and myriad of interactions. Differences in absorption and metabolism greatly affect bioavailability. Genotype, comorbidities, and an ever-changing background of anatomic and physiologic variations can alter pharmacokinetics drastically. In the absence of an unifying measure of immunosuppression, therapeutic drug monitoring has become the surrogate marker of optimal immunosuppression. But even this measure fluctuates widely. Some medications inhibit or induce cytochrome P450-dependent liver metabolism, whereas others affect P-glycoprotein transport. These interactions, among many others, result in highly unpredictable concentrations of immunosuppressants with inter- and intra-individual fluctuations that dictate close monitoring to adjust drug doses. For example, tacrolimus, a calcineurin inhibitor and a mainstay of solid organ transplantation, has a narrow therapeutic window and wide pharmacokinetic variability. Under-dosing of tacrolimus may result in under-immunosuppression and acute rejection. Over-dosing puts patients at risk of considerable neuro- and nephrotoxicity. In addition, tacrolimus binds to blood proteins, making its measurement very difficult. It is a substrate of both cytochrome P450 and P-glycoprotein, both with genetically variable expression levels in gut and liver. These factors combine to yield very poor inter- and intra-individual correlation between the dosing and the blood concentrations. In sum, its dosing is a clinical challenge. The lack of a consistent relationship between dose and blood concentration makes simplified calculations of pharmacokinetic parameters generally invalid. Frequent, individualized dosing is desired to administer tacrolimus safely.

The standard of care for administering tacrolimus at most transplantation centers is for a provider to adjust the dose according to a closely monitored trough blood concentration. This task is made more difficult given that the disease states of patient and their organ function fluctuate significantly during the first few months after transplantation. Determination of the next dose is based on daily measurements of tacrolimus level and hepatic and renal function. This approach is time and resource intensive and is unpredictable. As such it cannot be improved through conventional means. Patients frequently deviate from the targeted range and thus run the alternate risks of toxicity or rejection.

To address these dosing challenges, models have been developed to characterize pharmacokinetics in solid organ recipients with many covariates and uncertainty as to the importance of each covariate. These include population pharmacokinetic modeling, physiologically-based pharmacokinetic modeling, genetic modeling, and estimative forecasting. Using these approaches, adjustments to tacrolimus dose are overlayed upon highly variable early graft function, adjustments in other immunosuppression medications, adding and subtracting prophylactic and therapeutic antibiotic medications based upon presence of infections, and adjusting all medication doses based on their side-effects, such as nephrotoxicity and myelosuppression. These approaches also utilize intricate pharmacokinetic, genomic, and demographic data to estimate resulting drug levels. Attempts to increase the accuracy of prediction using these mechanism-dependent, model-based approaches involve implementing patient exclusion criteria. This is to prevent a virtually infinite range of confounding effects upon these mechanisms including genotypic, multi-drug, and other complex interactions. As such, a need exists for the ability to implement individualized responses to administered tacrolimus doses, particularly when co-infection is present, that would subsequently aid in reducing large fluctuations in drug levels and their attendant risks, as well as adapt to changes to patient regimens. This would facilitate arriving at and maintaining a targeted therapeutic range by comprehensively calibrating how each patient's treatment should be formulated.

With regards to combination therapy, drug combination performance can be dosage dependent and can be largely influenced by synergism, antagonistic, or additive drug interactions. Multi-drug dose modeling studies are able to examine the nonlinearity of drug-drug interactions. However, an universally-applicable platform that optimizes combination therapy using a system-level response and simultaneously addresses the relationship between input stimuli (drug administration) and phenotypic variation (biological response) across the cellular, tissue, and organism level was previously not yet realized. To address these challenges, the Feedback System Control (FSC) platform is developed for optimized combinatorial design, and the translation of FSC into a clinical setting resulted in an approach culminating in a powerful platform, termed PPM3′, which is a model-less and mechanism-independent approach that has been clinically-validated in this example to calibrate patient-specific responses to multi-drug therapy. PPM3′ of this example is not a pharmacogenomic or pharmacokinetic/predictive modeling approach as it utilizes phenotypic endpoints, which innately account for molecular and pharmacokinetic determinants without requiring complex modeling, to rapidly identify actionable treatment parameters that are optimized for an individual patient. In addition, PPM3′ of this example is not a systems biology platform. A number of systems biology studies have shown that cellular pathways form complex networks and that their collective dynamics drive phenotypic outcomes. Importantly, network dynamics cannot simply be explained by behavior of each network component and as such, therapeutically addressing several elements of a diseased network is important but virtually impossible to optimize using conventional strategies. This is due to the host of redundant signaling pathways, crosstalk, and compensatory and neutralizing mechanisms that comprise biological systems. In the case of post-operative transplant management, these complex interactions further substantiate the drive for adaptive treatment personalization that is mechanism-independent. Phenotype, as a function of the molecular/genetic mechanisms that serve as targets for therapeutic intervention, can be used to deterministically individualize treatment towards a broad spectrum of disorders.

Post-operative transplant patients undergo combination therapy with a diverse set of drugs and procedures that in addition to tacrolimus, includes mycophenolate (immunosuppressant), prednisone (anti-inflammatory), cotrimoxazole (antifungal), ganciclovir (antibiotic), and hemodialysis. This regimen is changed constantly in order to account for infection, inflammation, rejection episodes, and maintaining proper kidney function, among other complications. These patients respond uniquely to their respective, constantly-changing regimens. Some drugs are antagonistic for some and synergistic for others. PPM3′ reconciles patient-specific phenotypic responses to therapeutic intervention, and constructs a multi-dimensional quadratic phenotypic map that is dynamically adaptable to regimen changes. This is important towards providing actionable information about specific patients that allow for pre-emptive changes to their treatment or immediate re-calibration to mitigate against rejection episodes. Therefore, the objective of this prospective clinical study was to use PPM3′ to perform daily personalized optimization to identify tacrolimus dosages under highly variable treatment conditions. A retrospective clinical analysis was initially performed to show that the modulation of three drugs—tacrolimus, prednisone, and mycophenolate—was able to further improve convergence towards the target range and maintenance of tacrolimus trough levels. In the prospective clinical trial, four PPM3′-regulated patients were calibrated using three days of treatment (three data points) via standard of care to formulate a parabolic phenotypic map based on their respective phenotypic response (tacrolimus trough level). Four control patients were treated using standard of care for the entire duration of the study. After one month of treatment, patient-specific PPM3′ parabolic profiles mediated remarkable control over trough levels by guiding clinician dosing of tacrolimus alone. Control patients exhibited profoundly different outcomes. PPM3′ sensitivity profiles accounted for synergistic and antagonistic drug interactions and hemodialysis on an individualized level to preemptively prevent large deviations from target ranges. Perhaps one of the most significant findings from this example was that sudden regimen changes could be managed by shifting the PPM3′ parabolic profile and, in some cases, even one patient data point allows for patient re-calibration and re-convergence into target ranges. The immediately actionable nature of this approach and robust patient-specific management represent the definitive clinical implementation of personalized medicine.

Results

Retrospective FSC Optimization of Combination Therapy

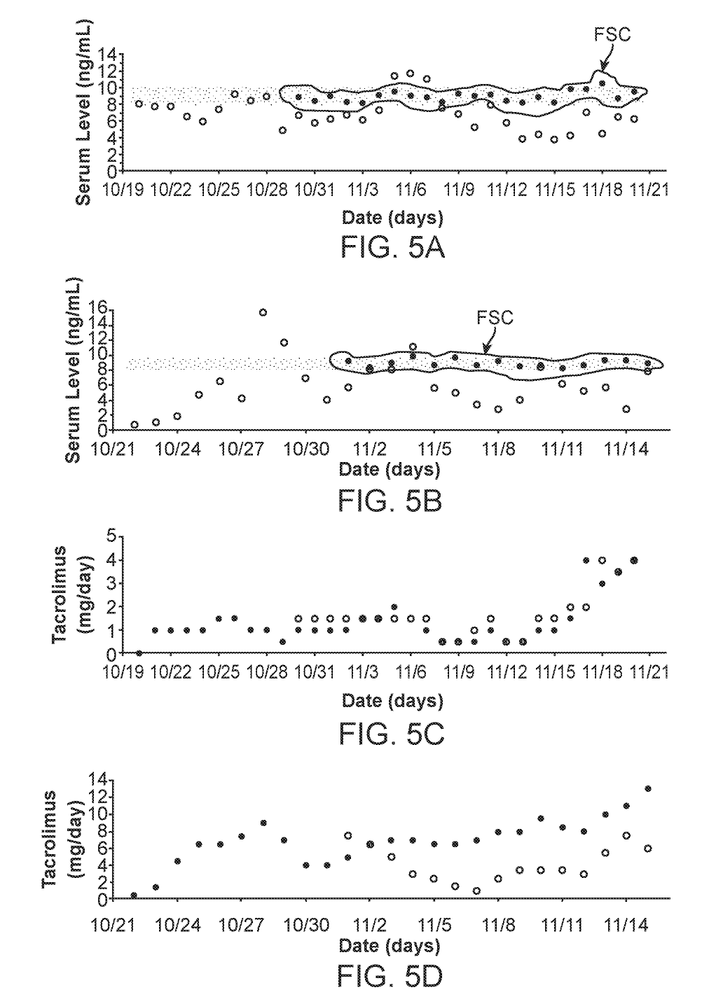

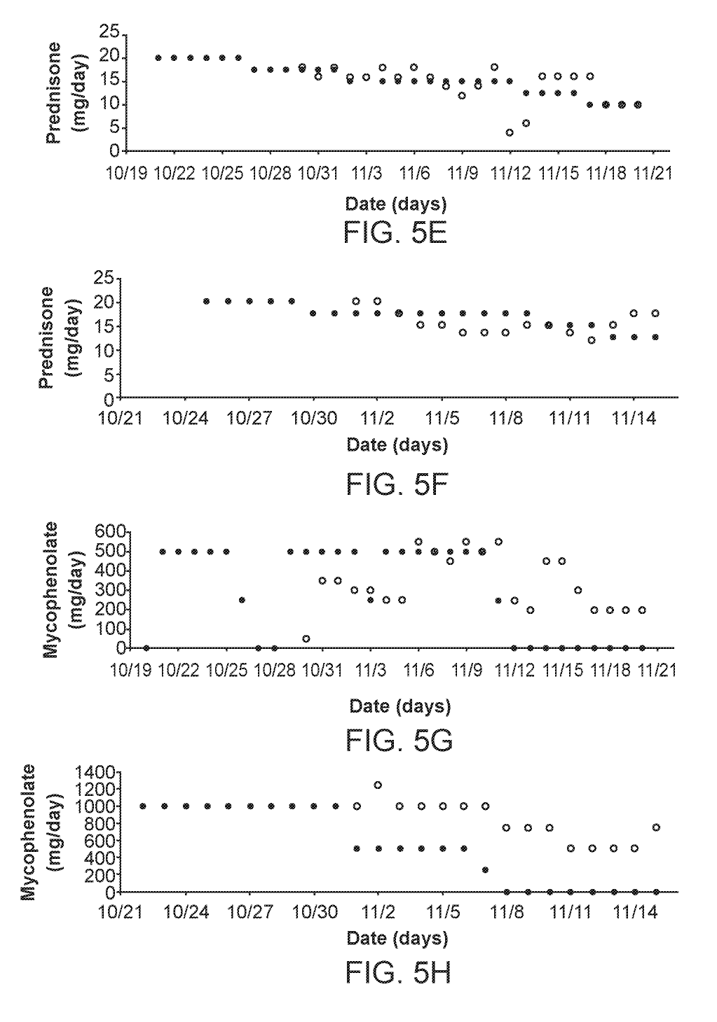

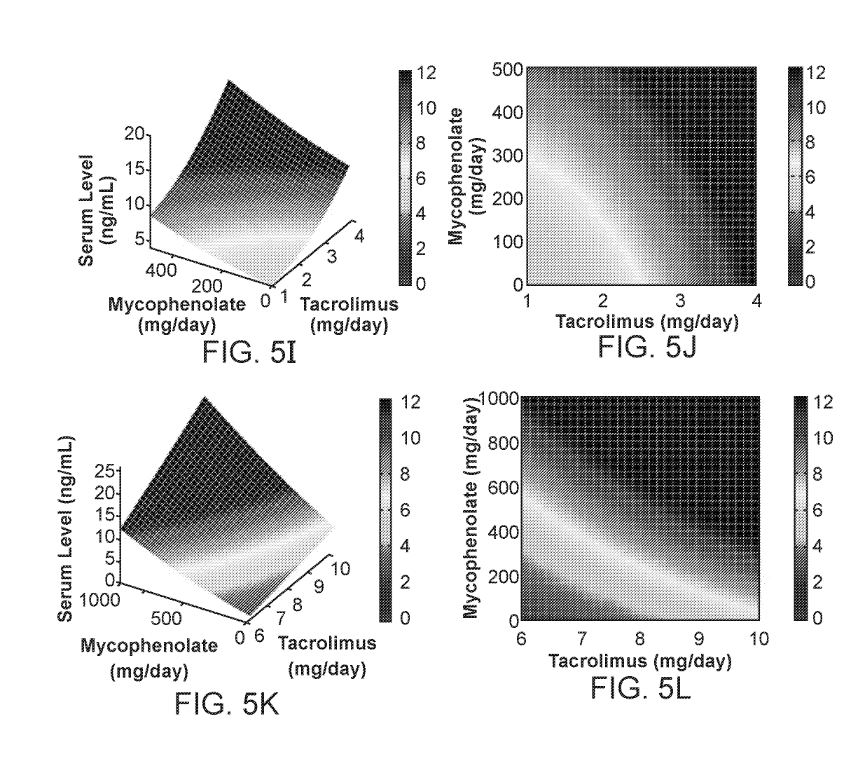

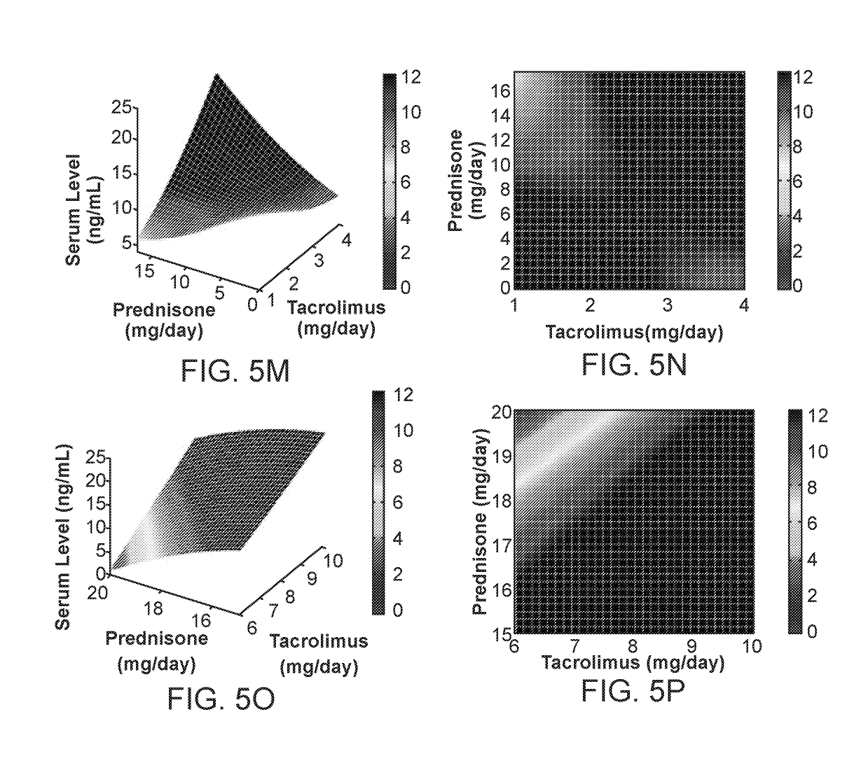

The initial validation of PPM3′ was performed using retrospective clinical analysis to determine the time-dependent multi-drug regimens to optimize immunosuppresive therapy. The compounds utilized included tacrolimus, mycophenolate, and prednisone. Target ranges were previously established for each patient, and their respective responses to the clinically-observed dynamic dosing of tacrolimus were used to identify personalized response constants. The patient trough level response to drug administration was used to calibrate the patient-specific constants to combination therapy. These constants were then used to prescribe dosing parameters for each drug that would maintain serum tacrolimus trough levels within the target ranges. FIG. 5A shows the serum tacrolimus trough levels as measured clinically and FSC-optimized trough levels for Patient A. Patient A was given a target range of 8-10 ng/ml based upon several factors including ethnicity, age, and basis of liver transplant (e.g., hepatitis cirrhosis, and so forth). The FSC-optimized trough levels converged into the target range at day 10 following a calibration period and remained within the target range for the duration of the analysis. Conversely, the clinically-observed tacrolimus trough levels frequently deviated from the target range and also exhibited multiple spikes, particularly during days 17-19 and 21 onward. FIG. 5B compares the FSC-optimized and standard of care-treated trough levels for Patient B. Again, following a calibration period, the FSC-optimized trough levels converged and remained within the prescribed range of 8-10 ng/ml while the conventionally-treated patients deviated permanently from the target range from day 15 onward. FIGS. 5C-H show the FSC-optimized tacrolimus, prednisone, and mycophenolate dosages per time point that varied profoundly from those that were given clinically. Importantly, the drug-dose ratio and each time point resulted in tacrolimus serum trough levels that remained in range for the duration of the study. FIGS. 5I and 5J show the three-dimensional (3-D) and two-dimensional (2-D) drug interaction maps, respectively for tacrolimus versus mycophenolate for patient A, which indicated a synergistic interaction due to a direct correlation between the tacrolimus and mycophenolate dosing for the trough level to converge within the target range of 8-10 ng/ml, where the highest drug dose combination was not required for the optimal outcome. FIGS. 5K and 5L show the 3-D and 2-D drug interaction maps, respectively for tacrolimus versus mycophenolate for patient B, which also indicated a synergistic interaction due to a direct correlation between the tacrolimus and mycophenolate dosing for the trough level to converge within the target range of 8-10 ng/ml. FIGS. 5M and 5N show 3-D and 2-D drug interaction maps, respectively for tacrolimus versus prednisone for patient A, which indicated a potentially antagonistic interaction due to an indirect correlation between the tacrolimus and prednisone dosing for the trough level to converge within the target range of 8-10 ng/ml. FIGS. 5O and 5P show the 3-D and 2-D drug interaction maps, respectively for tacrolimus versus prednisone for patient B, which indicated a synergistic interaction due to a direct correlation between the tacrolimus and prednisone dosing for the trough level to converge within the target range of 8-10 ng/ml.

The importance of the retrospective FSC analysis is based on its ability to simultaneously identify optimal drug-dose combinations for tacrolimus, mycophenolate, and prednisone at specific timepoints that allowed the tacrolimus trough levels to remain within the prescribed target ranges. Current clinical practice maintains prednisone on a specific weaning protocol whereby dosages are decreased in a stepwise fashion over time in increments of 2.5 mg from 20 mg to 17.5 mg, 15 mg, 12.5 mg, and eventually towards single digit values. Mycophenolate is prescribed in doses that remain the same over several days and eventually increase or decrease and then remain constant to provide immunosuppression support for tacrolimus. One of the reasons why these two compounds are administered according to specific guidelines is because current clinical practice does not possess a strategy that can rationally guide multi-drug, patient-specific dosing, particularly since the serum tacrolimus trough levels are sensitive to the co-administration of other immunosuppresants and antifungals in an individualized manner, as subsequent data will show. In other words, for some patients, prednisone will act antagonistically with tacrolimus, while for others, these two compounds will interact synergistically. This was shown in the retrospective analysis for patients A and B. Two important findings from this retrospective analysis emerged. Firstly, FSC-optimized retrospective analysis has shown that by modulating the dosages of tacrolimus as well as mycophenolate and prednisone, tacrolimus trough levels can be consistently maintained within target levels. This is a particularly important finding as these drugs are dosed in specific increments and as such, the dynamic dosing of all three drugs allows for much more versatility when it comes to rationally designing drug-dose combinations that circumvent the constraints of dosing increments. Secondly, it should be noted that patient A and B were also given several other drugs that are common following transplantation, such as ganciclovir, fluconazole, and cotrimoxazole, among others. The impact of these compounds on tacrolimus serum trough levels are implicitly built into the resulting clinical levels observed. Despite the large number of drugs given to these patients, retrospective FSC analysis harnessed the long study time window that innately included enough data points to account for and adapt to regimen changes. As such, FSC optimization rapidly targeted and maintained serum trough levels by modulating tacrolimus, prednisone, and mycophenolate. This signifies that the retrospective FSC study identified the lowest number of drugs to guide and optimize personalized immunosuppression, and, by allowing for FSC-prescribed multi-drug dose administration, the approach allows for remarkably robust targeting of patient-specific ranges to be achieved.

Personalized Immunosuppression Via FSC-Treated Patient ID5

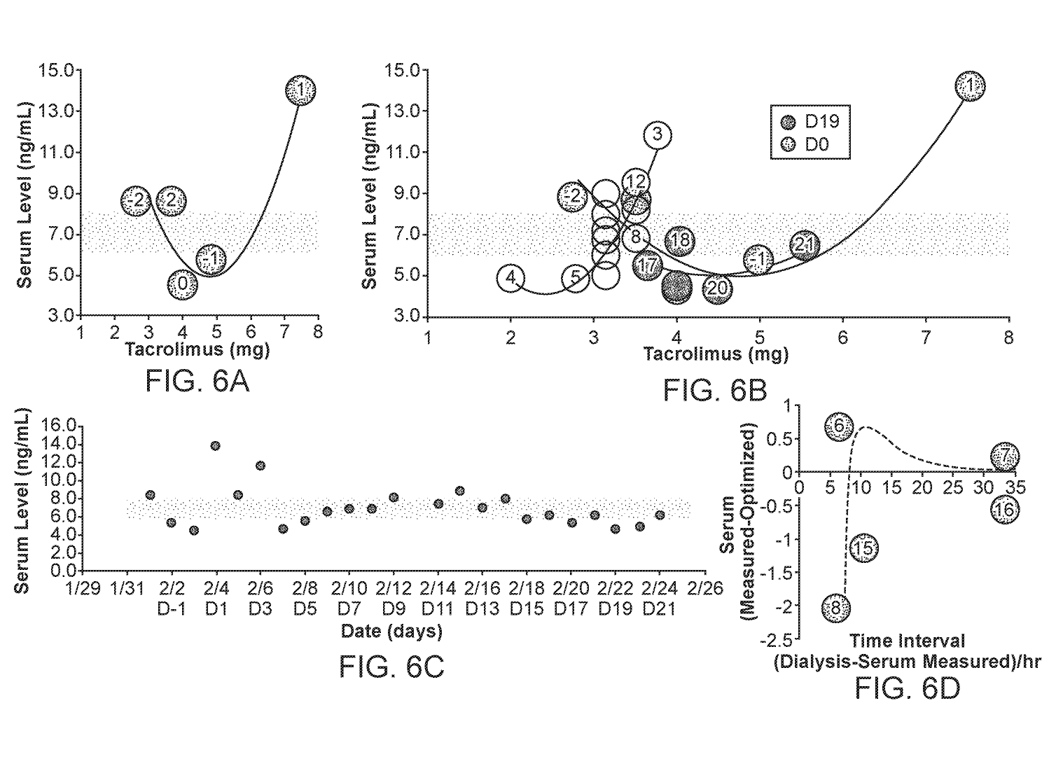

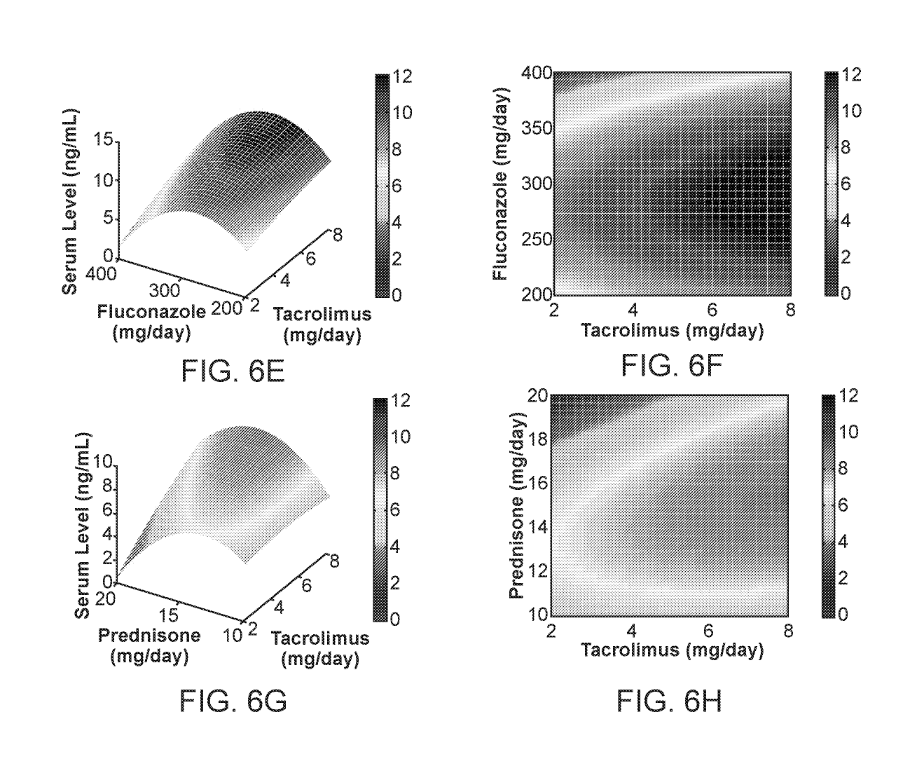

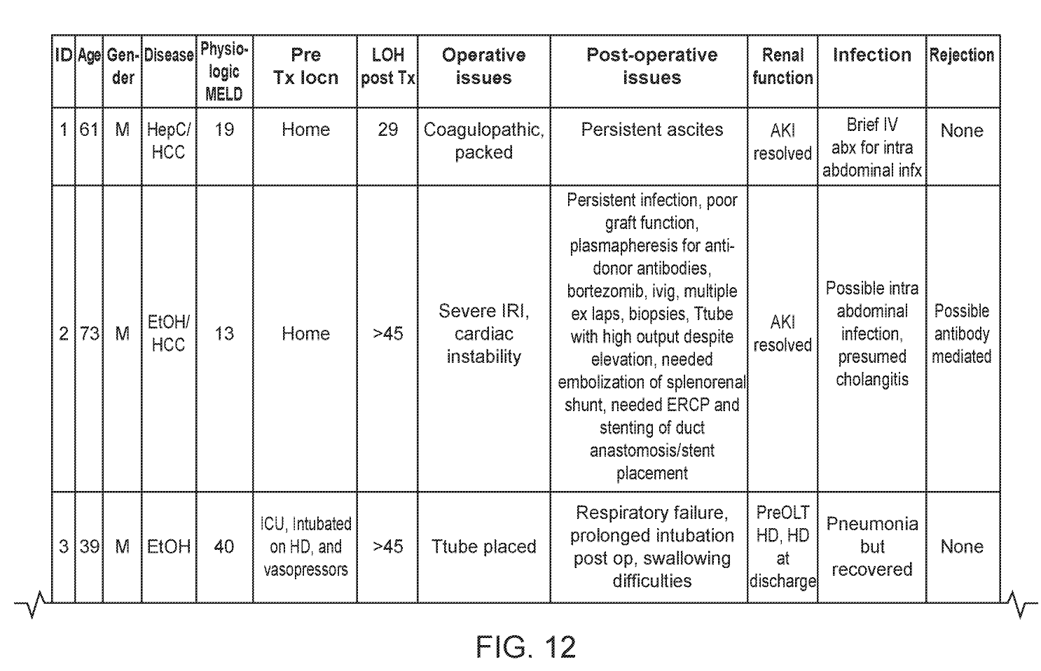

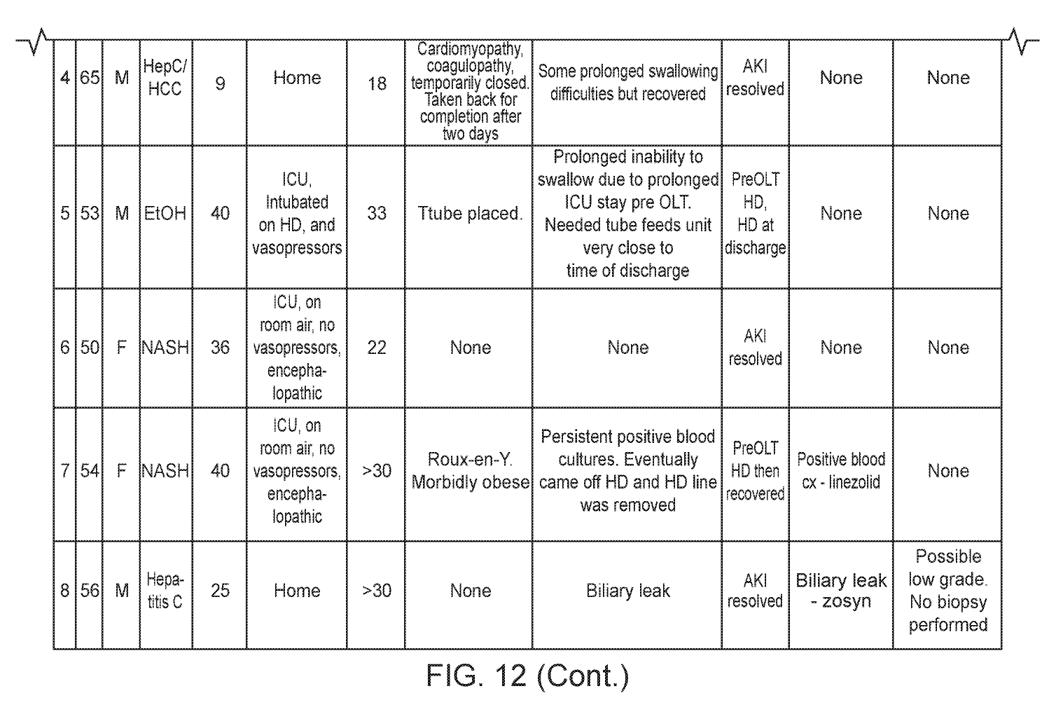

For the prospective clinical trial, the study allowed the modulation of tacrolimus dosing alone, despite several drugs being given to each patient during the course of treatment due to their diverse range of pre-operative and post-operative conditions (FIG. 12). Patient ID5 (Model of End-Stage Liver Disease (MELD) score of 40) was provided with clinical standard of care for a period of 10 days to serve as an initial calibration period. Drugs administered to ID5 included tacrolimus, prednisone, mycophenolate, fluconazole, cotrimoxazole, and ganciclovir, and several hemodialysis procedures were performed during the calibration and FSC treatment timeframe. Drug response constants specific to patient ID5 (target range 6-8 ng/ml) were used to construct 2-D parabolic phenotypic map with patient-specific quadratic phenotypic mapping constants represented as y=32.95−11.69x+1.22x². The constants were derived specifically for ID5 based upon the calibration period (FIG. 6A). This response surface was also used to prospectively identify tacrolimus doses while accounting for the dynamic nature of drug administration and co-infection as well as prophylactic therapy. Quadratic phenotypic mapping showed a remarkably robust command of ID5's tacrolimus serum trough levels, exhibited by the clustering of several trough readouts within the ID5 target range that corresponded accurately with the ID5-prescribed parabolic profile, resulting in a rational convergence in and around the target range for 10 days.

Frequent modifications were implemented in patient ID5's therapy regimen during the course of treatment. These included dosage increases or decreases, commencement or cessation of drug administration or hemodialysis, or change in drug medium from suspension to capsule. These regimen changes resulted in trough deviations that were addressed using a map translation process whereby the resulting trough level that deviated from the target range was used as a single data point to formulate a new projected parabolic surface with a slope that was correlated with that of the first parabolic plot. This translated curve was used to identify the next day's dosing prescription, and also served to obtain a second data point for the eventual construction of a new and re-optimized parabolic surface for the patient. FIG. 6B confirmed that the translated curve indeed re-established command of prospective patient management remarkably well with re-calibrated constants represented by y=26.39−18.45x+3.82x² and y=23.14−8.15x+0.92x². This finding showed that ID5 could be rapidly re-calibrated using this approach to re-converge within the target range (FIG. 6B). This allowed FSC to dynamically alter treatment which was particularly important as FSC treatment guidelines were prescribed on a daily basis for immediate clinical implementation. In addition, the retrospective clinical analysis shows that this is not just an empirical process, but rather confirms that the parabolic shift is a rational approach for patient-specific personalization of combination therapy during the course of dynamic drug administration. FIG. 6C shows the tacrolimus serum trough levels as a function of time that exhibited positive treatment outcomes with regards to converging towards and staying within or near the target range, and rapid re-calibration was attained. The initial deviations encountered (Days 1-3) were initiated when a tacrolimus dose of 7.5 mg was prescribed after an initial morning dose of 2 mg was administered to the patient. The resulting 5.5 mg administration in the evening, followed by an earlier than normal serum trough level taken the following morning resulted in a high trough level. Subsequent doses of 3.5 mg and 3.75 mg were given to re-calibrate this patient eventually to a steady dose of 3.25 mg that resulted in the rapid convergence of patient ID5 trough level regulation. It should be noted that in comparison to the retrospective studies, the prospective studies did not have the benefit of utilizing data from a long time window or the ability to make dose adjustments to all three drugs, all of which can be used to avoid target deviations altogether. However, the continued collection of retrospective data for patient sub-populations can be used to build a database to provide pre-emptive dosing compensation guidelines to predictively adjust dosing parameters in anticipation of regimen changes.

FIG. 6D represents an ID5-specific hemodialysis sensitivity and compensation plot, which showed that a minimum of ten hours between hemodialysis and the subsequent trough reading in order to prevent large deviations from anticipated trough levels. For example, two data points assessed within the ten hour window resulted in readings that were 0.5 ng/ml higher, or 2.0 ng/ml lower than the anticipated trough levels. The magnitude of hemodialytic impact upon trough levels was patient-specific. Therefore, this analysis allowed pre-emptive tacrolimus dose prescription adjustments to mitigate against large drops in measured trough levels due to fluid clearance. FIGS. 6E and 6F represent 3-D and 2-D drug response maps, respectively, for tacrolimus-fluconazole interactions, and FIGS. 6G and 6H represent 3-D and 2-D drug response maps, respectively, for tacrolimus-prednisone interactions. The divergence of conditions that resulted in target range convergence indicated potential antagonistic drug interactions between tacrolimus and mycophenolate, while the interaction between tacrolimus and prednisone appeared to be synergistic for ID5. This was further shown given that the highest drug-dose combinations were not required to maintain target trough levels. FIGS. 6A-H represent a comprehensive profile that was successfully constructed for each patient to mediate robust patient-specific control over a personalized immunosuppressive therapy regimen. Of note, patient ID5 was the first patient in the study to be discharged from the hospital.

Personalized Immunosuppression Via FSC-Treated Patient ID8

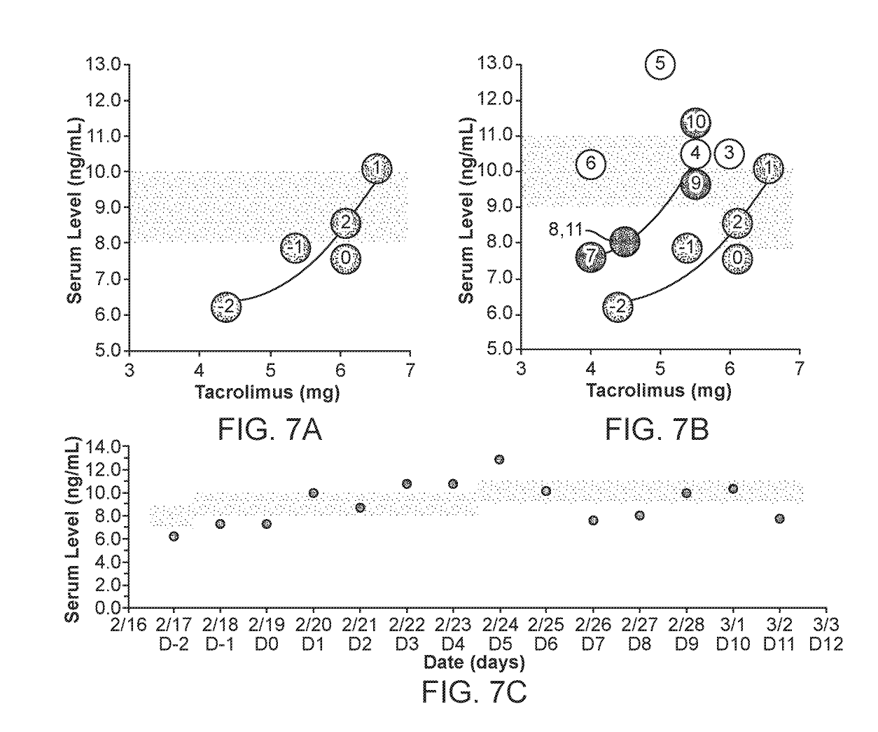

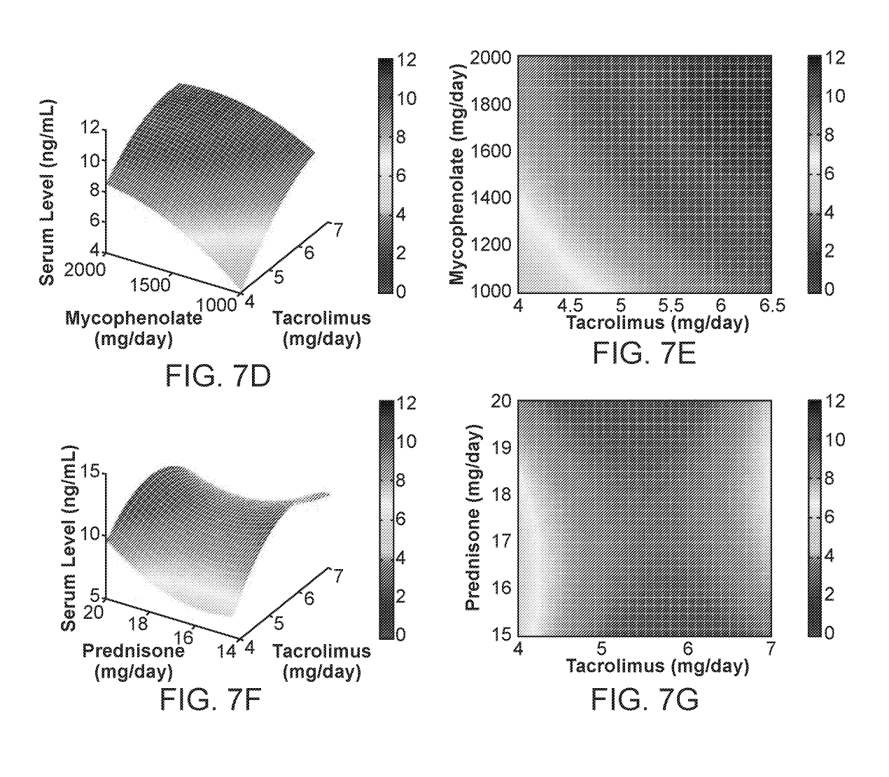

To provide an additional example of successful patient-specific command of tacrolimus trough levels under a different treatment regimen in comparison to that of ID5, parabolic phenotypic mapping was performed for patient ID8 (MELD score of 25). FIG. 7A shows the phenotypic map that was constructed specifically for ID8 based on the patient's calibration period. Patient ID8's regimen included tacrolimus, prednisone, mycophenolate, fluconazole, ciprofloxacin, cotrimoxazole, tenofovir and ganciclovir. Hemodialysis was not performed on ID8. This patient was given a target range of 8-10 ng/ml during most of the regimen, and the target was eventually amended to 9-11 ng/ml. FIG. 7A shows ID8's parabolic phenotypic map as represented by y=20.3−6.4x+0.73x², which differed from that of ID5, further demonstrating the patient-specific nature of treatment. Again, FSC optimization mediated robust control over maintaining ID8's trough levels within the target range. Due to several regimen changes that occurred in parallel, such as the continued step-wise increase of cotrimoxazole from 1000 mg eventually to 2000 mg daily, deviations from the target range resulted in response surface translations and re-formulation of patient-specific parabolic profiles that were re-calibrated for patient ID8, resulting in a revised phenotypic map represented by y=23−7.98x+1.03x² (FIG. 7B). Of note, Day 5 and Day 6 deviations were due to a daily cotrimoxazole dose increase to 2000 mg that, according to parabolic phenotypic mapping, would have resulted in marked increases in trough levels. To compensate for this anticipated increase, the tacrolimus dose was preemptively decreased from 5.5 mg, to 5 mg, and subsequently to 4 mg the following day. FSC was able to re-establish patient control for both target ranges of 8-10 ng/ml and 9-11 ng/ml that were determined for the patient (FIG. 7C). The drug response surfaces for tacrolimus versus mycophenolate and tacrolimus versus prednisone (FIGS. 7D-G) show that patient ID8 responded to therapy in a different manner than ID5, and target ranges were reached using a regimen specific to this patient, further substantiating the drive for personalized treatment.

Immunosuppression Via Clinical Standard of Care-Control Patient ID6

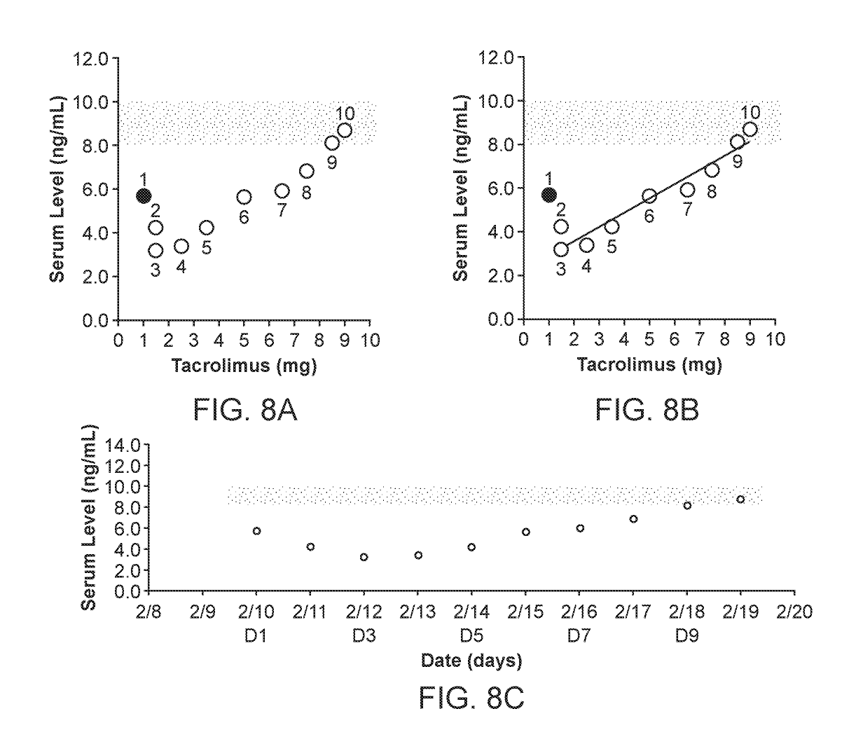

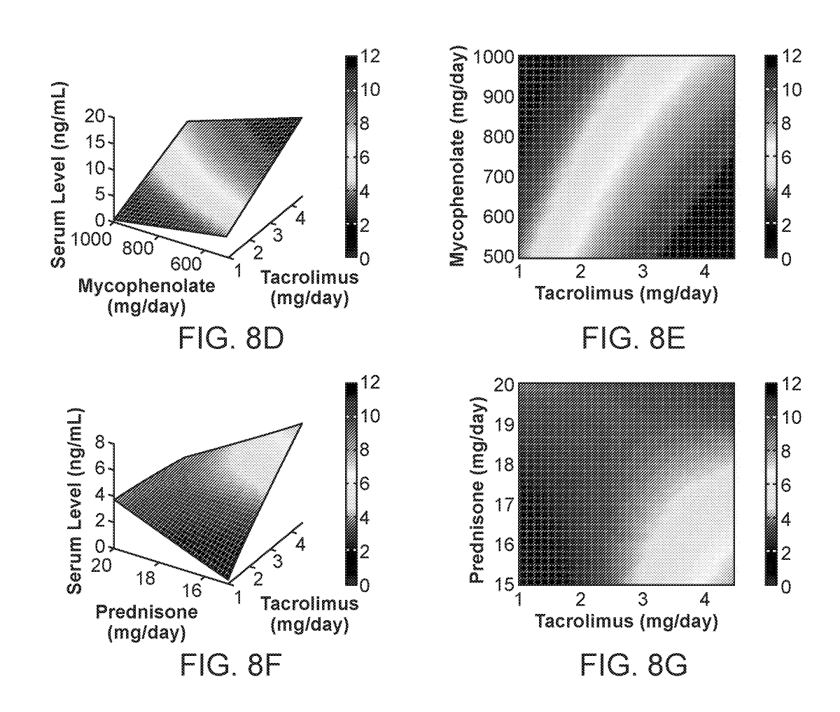

To compare the results of FSC-directed parabolic phenotypic mapping with control patient treatment, patient ID6's tacrolimus dosing regimen was analyzed. A profound difference in the immunosuppression regimen and markedly different patient response was observed for ID6 (MELD score of 36) in comparison to FSC-regulated patients. FIG. 8A shows ID6's phenotypic map. The level of scatter that represented the tacrolimus serum trough levels made it such that parabolic phenotypic mapping could not be completed for this phase of treatment. Tacrolimus was initially administered at dosages of 1.5-4 mg due to dynamic post-operative target ranges such 5-7 ng/ml, 6-8 ng/ml and 7-9 ng/ml, resulting in trough levels ranging from 2.7-6.9 ng/ml. Once a steady target range of 8-10 ng/ml was established, however, tacrolimus dosing was increased to 6 mg, suddenly resulting in a trough level of 13.4. This sharp increase resulted in a sudden drop in tacrolimus dosing to 1 mg. This ultimately caused the trough levels to drop well below the target range and to consistently reside at levels that were over 2 ng/ml below the target threshold. In the absence of a parabolic phenotypic map to guide patient therapy, a step-wise increase in tacrolimus dosing was eventually employed, most notably during days 12-19 (FIG. 8B). During this time period, tacrolimus dosages ranging 1.5 mg to 9 mg were given incrementally on a daily basis. This regimen did not reach the target range of 8-10 ng/ml until day 19 (trough level: 8.1 ng/ml), and subsequently deviated from the target range again. In sum, patient ID6 trough levels were outside of the target range during 91% of the patient's treatment (FIG. 8C). Construction of a phenotypic map for days 12-19 was attempted via retrospective analysis. Of note, due to days 12-19 trough data being out of range for nearly the entire duration, parabolic phenotypic mapping was again precluded (FIG. 8B). As expected, days 12-19 analysis showed a clear linear trend which was shown as y=0.653x+2.27 (R²=0.93). Given the absence of parabolic phenotypic mapping, gradual titration was ultimately incapable of systematically converging into the target range. FIGS. 8D and 8E show the patient ID6 drug response maps for tacrolimus versus mycophenolate, and FIGS. 8F and 8G represent tacrolimus versus prednisone interactions, respectively. In comparison to patients ID5 and ID8, patient ID6 exhibited a patent-specific and synergistic interaction between these compounds represented by the observation that the highest drug dosages were not required for trough level targeting.

FSC Systematically Identifies Patient-Specific Response to Regulate Phenotype

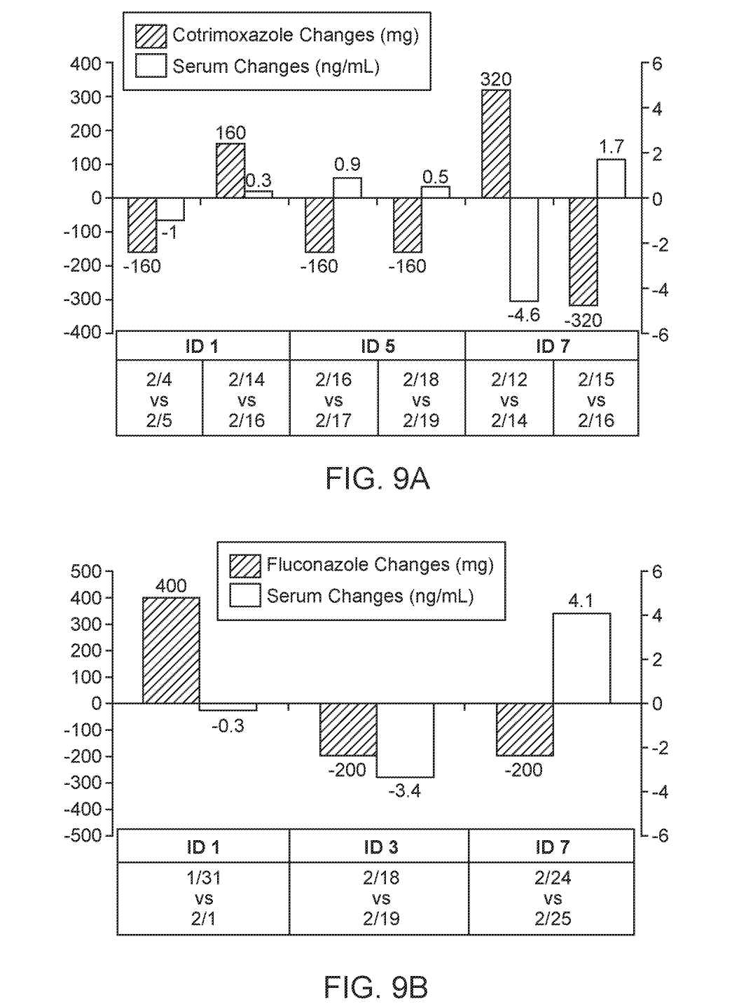

In addition to the application of parabolic phenotypic mapping towards patient-specific drug interaction assessment, individualized drug sensitivity levels were identified where the introduction of prophylactic antibiotics impacted resulting trough levels (FIG. 9). The ability to pinpoint these sensitivity levels played a role in compensating for downstream administration protocols to truly personalize treatment. Repeated cycles of cotrimoxazole and fluconazole administration, and protocol-implemented increases or decreases in mycophenolate dosing resulted in patient-specific trends for trough level response. For example, patient ID1's cotrimoxazole administration resulted in a directly correlated serum trough level response. Specifically, a 160 mg decrease in cotrimoxazole dosing resulted in a serum trough decrease of 1 ng/ml. In contrast, the same decrease in cotrimoxazole in patient ID5 resulted in a 0.9 ng/ml trough level increase, an inversely correlated relationship. Similarly, a 320 mg increase in cotrimoxazole resulted in a 4.6 ng/ml decrease in serum trough levels for patient ID7. For fluconazole administration, patient ID3 exhibited a direct correlation with serum trough levels, where a 200 mg decrease in dose resulted in a 3.4 ng/ml drop in trough level. In contrast, a 200 mg decrease in patient ID7 resulted in a 4.1 ng/ml increase in trough level. The data showed that given the broad range of patient-specific responses to regimen modification, personalized treatment is imperative for optimal immunosuppressive therapy (Tables 2 and 3).

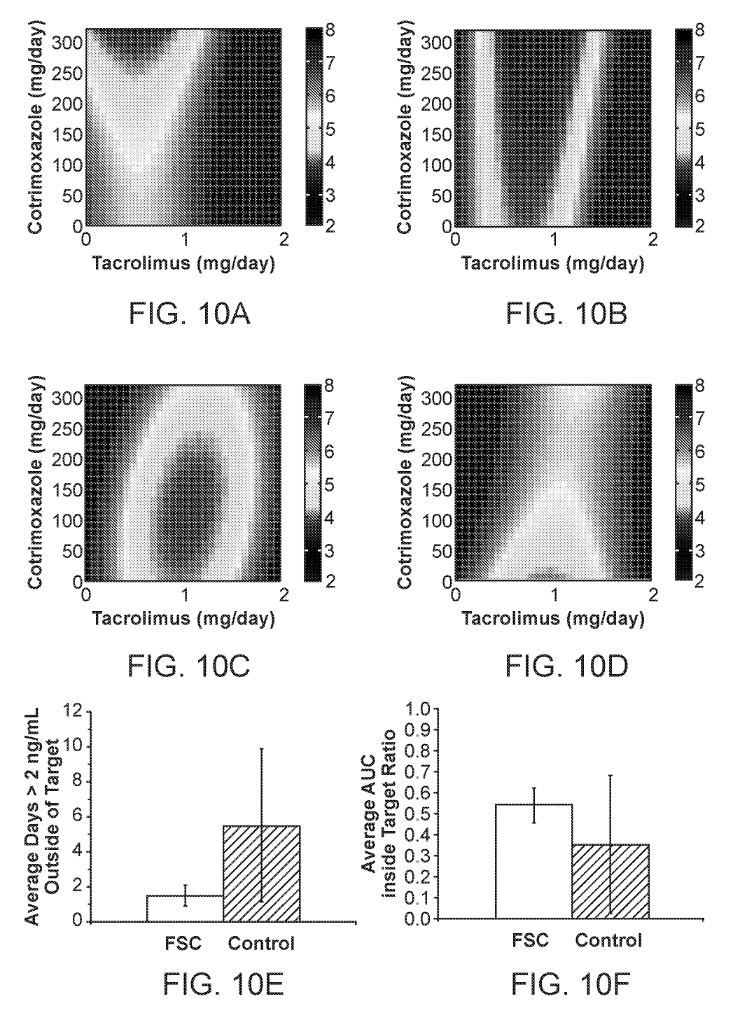

To further show that FSC can properly account for patient response to mediate the personalization of immunosuppression, drug response mapping was conducted over time to assess the synergistic or antagonistic nature of drug-drug interactions and their impact on tacrolimus serum trough levels. FIGS. 10A-D show a drug response map over time for patient ID7 (FSC-treated) that correlates tacrolimus and cotrimoxazole dosing with tacrolimus serum trough levels. During the course of treatment, the evolution of the response map shows that the relationship between the two drugs changes from being synergistic to antagonistic as represented by the divergent drug dosing conditions that allow trough levels to reside within the target range. Patient ID3 (control) serves as another example where FSC-based calibration of drug sensitivity and treatment personalization were able to definitively account for deviation from the target range. For example, following a period of steady tacrolimus dosing levels that also resulted in trough readings that were within the assigned range of 6-8 ng/ml, cessation of cotrimoxazole administration on day 29 resulted in a substantial tacrolimus trough increase from 6.8 ng/ml to 8.8 ng/ml despite a tacrolimus dose that had not increased. This was followed by a continued increase to 9.4 and 9.7 ng/ml despite the same dose (4 ng/ml) of tacrolimus being prescribed for 5 straight days. Following a tacrolimus dose reduction to 3 ng/ml, the trough level then dropped to 8.6 ng/ml. However, following a 6.6 ng/ml reading in the assigned range the next day, the trough level then fell out of range to 5.9 ng/ml, which was likely due to a hemodialysis treatment from which patient sensitivity was not derived or accounted for in the subsequent dose prescription.

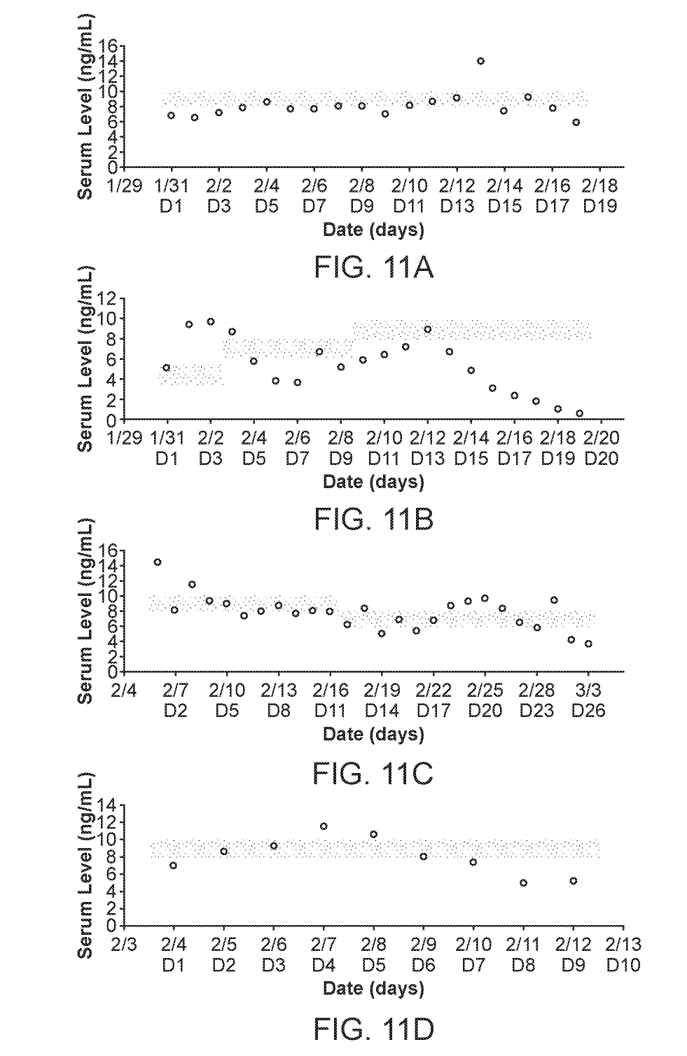

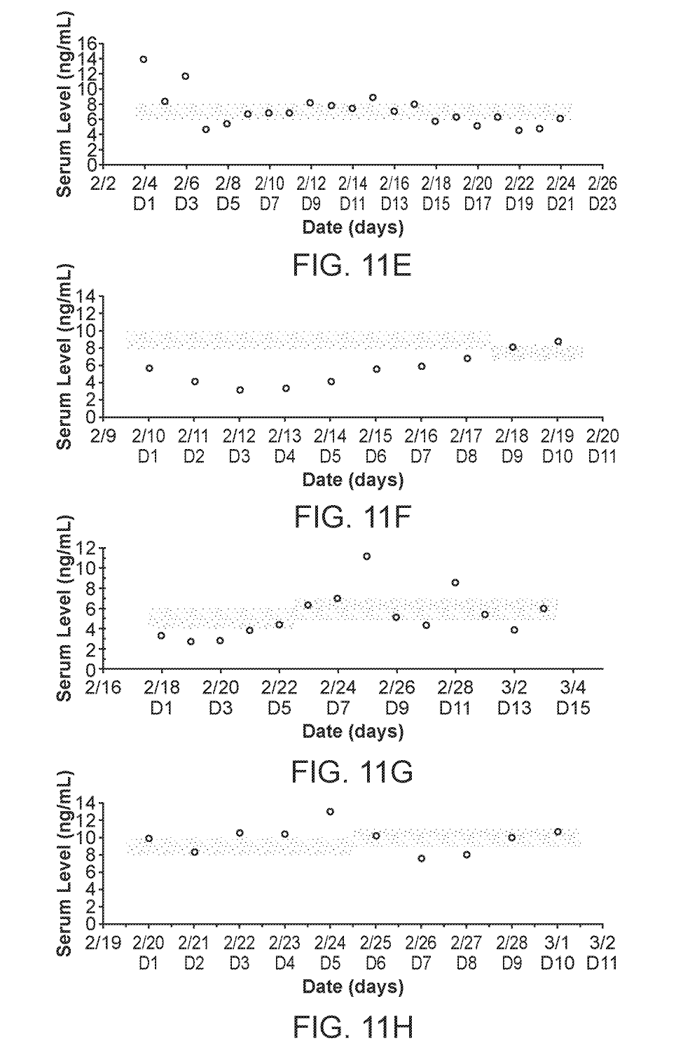

FSC treatment outcomes were directly compared with those mediated by control therapy. Firstly, the Institutional Review Board (IRB) that governed the implementation of this trial stated that deviations from target trough levels that exceed 2 ng/ml would involve additional physician evaluation and a potential reversion to standard of care implementation for tacrolimus dosing. FIGS. 10E and 11 show that the FSC-treated patients (mean±SD: 1.5±0.58) averaged less days of major deviation greater than 2 ng/ml from the target range compared to control patients (mean±SD: 5.5±4.4). Importantly, the lower standard deviation observed with the FSC-treated patients compared to control patients also showed that FSC provides more tightly regulating patient trough levels compared to control patients. In sum, FSC-treated patients experienced a total of 6 trough readings that were greater than 2 ng/ml out of range, while control patients had 22 trough readings that were greater than 2 ng/ml out of range. FIGS. 10F and 11 show that the FSC-treated patients (mean±SD: 0.54±0.08) had a larger area-under-the-curve (AUC) within their respective target ranges compared to control patients (mean±SD: 0.35±0.33). Again, the lower standard deviation observed with the FSC-treated patients compared to control patients further confirmed an improved ability to regulate patient outcomes via FSC. In sum, parabolic phenotypic mapping and direct FSC to control comparisons have indicated that personalized immunosuppression mediates improved treatment outcomes over clinical standard of care.

Discussion

This clinical study represents an importance advance in the clinical implementation of personalized medicine as it has demonstrated the use of phenotype to converge towards desired patient endpoints. Importantly, this can be accomplished in a mechanism-independent and model-less fashion. Furthermore, this clinical study has substantiated that the basis of mechanism-independent, personalized medicine can be explained by the following analysis that holds for biological complex systems.

To further illustrate the foundation of PPM3′, the physiology of a biological complex system can be represented by F(S), where S comprises the genomic and proteomic networks that govern the steady or diseased states of biological systems. The phenotype (e.g., trough levels, tumor size, cell viability, pathogen loads, and so forth) of a perturbed biological complex system under therapeutic intervention can be expressed as a function, F(S′,C), where S′[p] are the mechanisms in the aberrant networks perturbed by the disease causing agents, p. The therapeutic intervention, C, is composed of m types of drugs, d, and n concentrations each, C[d,x]. According to the Taylor expansion in mathematics, F(S′,C) can be related to the diseased biological complex system prior to therapeutic intervention, F(S) as:

$$F ( {S}^{′} , C ) = F ( S ) + {\sum }_{i} {a}_{i} {x}_{i} + {\sum }_{{ii}^{′}} {b}_{{ii}^{′}} {x}_{i} {x}_{{i}^{′}} + higher order terms$$

Due to the sheer complexity of the genomic and protein-based mechanistic networks of a biological complex system, the explicit function of F(S) and the function of diseased system under therapy, F(S′,C) are unknowns. In order to identify the mechanisms, S and S′, one would have to inverse the unknown functions which are comprised of a prohibitory large number of parameters. This would be a formidable task.

On the other hand, it has been experimentally demonstrated that the higher order terms are much smaller than the first and second order terms. Therefore, by moving the F(S) term to the left side of the equation and neglecting the higher order terms, the right hand side of the equation can then represent the efficacy of a combination therapy as:

$$E ( {S}^{′} , C ) = F ( {S}^{′} , C ) - F ( S ) \approx {\sum }_{i} {a}_{i} {x}_{i} + {\sum }_{{ii}^{′}} {b}_{{ii}^{′}} {x}_{i} {x}_{{i}^{′}}$$

The efficacy, or in this case, the difference between the two unknown equations, can be expressed by a quadratic algebraic series. This serves as the basis of quadratic phenotypic mapping that mediates the implementation of personalized medicine as a calibration process between the drug-dose inputs and the phenotypic outputs of a specific patient. For example, if three drugs are used in the therapy, ten tests of drug-dose variations can then determine the ten constants to allow quadratic phenotypic mapping. From this mapping, an optimal drug-dose combination for a specific patient from a very large combinatorial drug-dose parameter space can then be rapidly and definitively identified.

The introduction of retrospective clinical analysis using FSC in this example has shown that the modulation of three core compounds that are used during immunosuppression—tacrolimus, prednisone, and mycophenolate—can markedly improve the speed of convergence into and maintenance within patient-specific target ranges. Of note, retrospective clinical analysis carries the benefit of having a long time window during which an adequate number of data points for each change in regimen can be used to effectively regulate trough levels and prevent trough level spiking events. Retrospective FSC clinical analysis will likely serve as an effective protocol to identify drug interactions and other factors that influence tacrolimus serum trough levels that are sub-population-specific. This may allow pre-emptive adjustments to be made when regimen changes are anticipated to prevent acute rejection events when trough levels end up being too low or neuropathological side effects when trough levels are too high.

Prospective parabolic phenotypic mapping showed that even with the presence of co-infection, prophylaxis, and hemodialysis, a robust command of patient trough levels was achieved. When target range deviations occurred due to unforeseen or major revisions to treatment regimens, FSC mediated systematic re-calibration and convergence into target ranges. This is a markedly different approach than the clinical standard of care that conventionally relies on titration or incremental dosing. Control patients ID6 and ID3 served as clear indicators that these approaches were unable to mediate successful patient trough level control. Therefore, retrospective parabolic phenotypic mapping was precluded due to, either, or both, large deviations from target trough levels and persistent trough levels that were outside of the target levels. In summary, this example has shown that clinical personalized medicine can be realized in a mechanism-independent and model-less fashion, particularly for indications that involve actionable and dynamic treatment strategies.

An important attribute of this prospective clinical trial was the level of interaction between the clinical and optimization teams. The foundation of this trial was the implementation of daily dosing recommendations that were identified via parabolic phenotypic mapping. Due to the frequency of reporting and treatment involved in this study, a streamlined process whereby a comprehensive report of the morning clinical readouts as well as potential downstream treatment modifications were methodically provided to the FSC optimization team based upon which the tacrolimus dosing prescriptions were provided to the clinical team in the evening. This seamless integration of clinical and FSC expertise served as a foundation for the success of this trial. Further improvements involve the construction of population-specific templates to scale the process of clinical phenotypic personalized medicine so that a broad community of patients with a spectrum of diagnoses will benefit from mechanism-independent treatment.

Materials and Methods

Retrospective Clinical Analysis

Retrospective clinical analysis was performed under IRB#14-001682 approved by the UCLA Institutional Review Board. Discharged liver transplantation patient data, such as serum level and drug regimen dosages, were obtained for analyses for the retrospective study. In order to project optimum dosages, a 2^{nd} order polynomial fit for each patient was made from linear regression with covariates that included all the drugs given to the patient: tacrolimus, prednisone, mycophenolate, and so forth. In this process, the inclusion of all the drugs as covariates allowed better representation of each patient's data and examination of the effects of drug-drug interactions within each patient's constructed map. Minimum values based on the difference between the individual patient's serum level reading and a target serum level during various small time windows were collected, and these were entered into the globally fitted map of each patient in order to find the optimum dose for that specific patient.

Prospective Clinical Treatment Protocol

This prospective trial was conducted under IRB#14-001682, IRB#14-001682-AM-00001, and IRB#14-001682-AM-00002 approved by the UCLA Institutional Review Board. Following liver transplant, patients were started on a regimen of tacrolimus, mycophenolate, and methylprednisolone. According to protocol, methylprednisolone dosing was tapered using a 1000-150-210-120-80-40-20 mg regimen. Methylprednisolone was subsequently changed to prednisone given at 20 mg which was commonly followed with a tapering protocol. Mycophenolate dosing was administered according to protocols determined by the clinical team. Serum trough levels were taken between 4:00-6:00 AM daily, and a first dose of tacrolimus was administered between 5:00 AM-6:00 AM daily, following the serum trough recording. The clinical team obtained the daily treatment regimen details including drugs already administered, drugs to be administered, hemodialysis to be performed, co-infections, and planned prophylaxis and sent an updated clinical chart for each patient to the FSC team. Following parabolic phenotypic mapping analysis, the FSC team sent the suggested total daily tacrolimus administration information to the clinical team prior to the administration of a second evening dose.

Prospective Clinical Analysis Via Parabolic Phenotypic Mapping

Serum levels, drug regimen dosages, and other events such as hemodialysis were obtained every morning prior to analyses. In order to project optimum dosages, a 2^{nd} order polynomial fit for each patient was made from linear regression with mainly two variables, such as trough concentration and tacrolimus dosing amount, and it was used with at least three prior data points for the specific patient. Additionally, the effect and degree of drug-drug interactions upon individual patients obtained during the prospective study were considered when predicting the better dosage regimen.

Statistical Analysis

Statistical analyses were performed by analyzing the accuracy of correlating 2^{nd} order polynomial mapping to the patients' data since patient-specific treatment was conducted in this study. This was accomplished using the coefficient of determination, or R-squared analysis. Values were used to correlate clinical and regression data. Where relevant, statistical significances of all the generated maps for the data analyses were determined using a t-test at α=0.05 level of confidence.

TABLE 2
Summary of patient-specific antibiotic and antifungal responses.
A summary correlation between tacrolimus serum trough
levels to therapeutic co-administration is shown.

| | | | | Serum |
|---|---|---|---|---|
| Drug | Patient | Date vs Date | Change | Result |
| Amikacin and | ID 5 | 1/31 vs 2/1 | −400 (A) | +1.4 |
| Ganciclovir | | | −110 (G) | |
| Amikacin and Linezolid | ID 3 | 2/7 vs 2/8 | −575 (A) | +3.4 |
| | | | −1200 (L) | |
| Amikacin and Piperacillin- | ID 3 | 2/2 vs 2/3 | −150 (A) | +1.7 |
| Tax | | | −6750 (P) | |
| Ciproflox | ID 3 | 1/31 vs 2/1 | −400 | 0 |
| Cotrimoxazole | ID 1 | 2/4 vs 2/5 | −160 | −1 |
| | ID 1 | 2/14 vs 2/16 | +160 | +0.3 |
| | ID 5 | 2/16 vs 2/17 | −160 | +0.9 |
| | ID 5 | 2/18 vs 2/19 | −160 | +0.5 |
| | ID 7 | 2/12 vs 2/14 | +320 | −4.6 |
| | ID 7 | 2/15 vs 2/16 | −320 | +1.7 |
| Fluconazole | ID 1 | 1/31 vs 2/1 | +400 | −0.3 |
| | ID 3 | 2/18 vs 2/19 | −200 | −3.4 |
| | ID 7 | 2/24 vs 2/25 | −200 (F) | +4.1 |
| Linezolid | ID 7 | 2/23 vs 2/24 | −600 | +0.7 |

TABLE 3
Summary of patient-specific anti-inflammatory and immunosuppressant
responses. A summary correlation between tacrolimus serum
trough levels to therapeutic co-administration is shown.

| Drug | Patient | Date vs Date | Change | Serum Result |
|---|---|---|---|---|
| Methylpred | ID 8 | 2/11 vs 2/12 | −120 | −1.3 |
| Mycophenolate | ID 6 | 2/9 vs 2/13 | +1000 | −7.4 |
| | ID 8 | 2/18 vs 2/23 | +500 | +2.7 |
| Prednisone | ID 3 | 2/5 vs 2/6 | −2.5 | +6.5 |
| | ID 5 | 2/5 vs 2/11 | −2.5 | −1.5 |
| | ID 5 | 2/14 vs 2/16 | −2.5 | −0.4 |
| | ID 5 | 2/14 vs 2/18 | −2.5 | −1.7 |
| Prednisone and | ID 5 | 1/30 vs 2/3 | −2.5 (P) | −2.9 |
| Ganciclovir | | | −10 (G) | |
| | ID 7 | 2/17 vs 2/19 | −2.5 (P) | −2.6 |
| | | | +125 (G) | |

While certain conditions and criteria are specified herein, it should be understood that these conditions and criteria apply to some embodiments of the disclosure, and that these conditions and criteria can be relaxed or otherwise modified for other embodiments of the disclosure.

As used herein, the singular terms “a,” “an,” and “the” include plural referents unless the context clearly dictates otherwise. Thus, for example, reference to an object can include multiple objects unless the context clearly dictates otherwise.

As used herein, the terms “substantially” and “about” are used to describe and account for small variations. When used in conjunction with an event or circumstance, the terms can refer to instances in which the event or circumstance occurs precisely as well as instances in which the event or circumstance occurs to a close approximation. For example, when used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

While the disclosure has been described with reference to the specific embodiments thereof, it should be understood by those skilled in the art that various changes may be made and equivalents may be substituted without departing from the true spirit and scope of the disclosure as defined by the appended claims. In addition, many modifications may be made to adapt a particular situation, material, composition of matter, method, operation or operations, to the objective, spirit and scope of the disclosure. All such modifications are intended to be within the scope of the claims appended hereto. In particular, while certain methods may have been described with reference to particular operations performed in a particular order, it will be understood that these operations may be combined, sub-divided, or re-ordered to form an equivalent method without departing from the teachings of the disclosure. Accordingly, unless specifically indicated herein, the order and grouping of the operations is not a limitation of the disclosure.

What is claimed is:

1. A method comprising:
deriving an initial phenotypic map for a patient subjected to an initial therapeutic regimen including a first drug, based on values of a therapeutic outcome for the patient and corresponding values of a dose of the first drug administered to the patient;
following a change from the initial therapeutic regimen to an updated therapeutic regimen, receiving an updated value of the therapeutic outcome for the patient subjected to the updated therapeutic regimen;
re-calibrating the initial phenotypic map according to the updated value of the therapeutic outcome; and
using the re-calibrated phenotypic map, identifying a value of the dose of the first drug to be administered to the patient subjected to the updated therapeutic regimen.

2. The method of claim 1, wherein the initial phenotypic map is a quadratic function relating the therapeutic outcome and the dose of the first drug.

3. The method of claim 1, wherein the change from the initial therapeutic regimen to the updated therapeutic regimen includes an increase or a decrease in a dose of a second drug included in the initial therapeutic regimen.

4. The method of claim 1, wherein the change from the initial therapeutic regimen to the updated therapeutic regimen includes adding a second drug to the initial therapeutic regimen.

5. The method of claim 1, wherein the change from the initial therapeutic regimen to the updated therapeutic regimen includes removing a second drug included in the initial therapeutic regimen.

6. The method of claim 1, wherein re-calibrating the initial phenotypic map includes shifting the initial phenotypic map according to the updated value of the therapeutic outcome.

7. The method of claim 1, wherein re-calibrating the initial phenotypic map includes shifting the initial phenotypic map so as to intersect the updated value of the therapeutic outcome while maintaining a shape of the initial phenotypic map.

8. A method comprising:
deriving an initial phenotypic map for a patient subjected to an initial therapeutic regimen including a first drug and a second drug, based on values of a phenotypic output for the patient and corresponding values of doses of the first drug and the second drug;
following a change from the initial therapeutic regimen to an updated therapeutic regimen; receiving an updated value of the phenotypic output for the patient subjected to the updated therapeutic regimen;
re-calibrating the initial phenotypic map according to the updated value of the phenotypic output; and
using the re-calibrated phenotypic map, identifying values of the doses of the first drug and the second drug for the patient subjected to the updated therapeutic regimen.

9. The method of claim 8, wherein the initial phenotypic map is a quadratic function relating the phenotypic output and the doses of the first drug and the second drug.

10. The method of claim 9, wherein deriving the initial phenotypic map includes fitting the values of the phenotypic output and the corresponding values of the doses of the first drug and the second drug with respect to the quadratic function.

11. The method of claim 8, wherein the change from the initial therapeutic regimen to the updated therapeutic regimen includes an increase or a decrease in a dose of a third drug included in the initial therapeutic regimen.

12. The method of claim 8, wherein the change from the initial therapeutic regimen to the updated therapeutic regimen includes adding a third drug to the initial therapeutic regimen.

13. The method of claim 8, wherein the change from the initial therapeutic regimen to the updated therapeutic regimen includes removing a third drug included in the initial therapeutic regimen.

14. The method of claim 8, wherein re-calibrating the initial phenotypic map includes shifting the initial phenotypic map so as to intersect the updated value of the phenotypic output while maintaining a shape of the initial phenotypic map.

15. A method comprising:
for each patient of a group of patients,
deriving parameters of an individual phenotypic map for the patient administered with at least one drug, based on values of a therapeutic outcome for the patient and corresponding values of a dose of the drug administered to the patient; and
averaging the parameters across the group of patients to derive averaged parameters of a population level phenotypic map.

16. The method of claim 15, wherein deriving the parameters of the individual phenotypic map includes fitting the values of the therapeutic outcome and the corresponding values of the dose of the drug with respect to a quadratic function relating the therapeutic outcome and the dose of the drug.

17. The method of claim 15, further comprising:
using the population level phenotypic map, identifying an optimized value of the dose of the drug.
